(19) United States
(12) Patent Application Publication
(10) Pub. No.: US 2026/0002944 A1
(43) Pub. Date: Jan. 1, 2026

(54) BIOMARKERS OF FIBROSIS AND USES THEREFOR

(71) Applicant: Griffith University, Nathan (AU)

(72) Inventors: Chamindie Punyadeera, Nathan (AU); Darrell Crawford, Nathan (AU)

(21) Appl. No.: 18/869,131

(22) PCT Filed: May 29, 2023
(86) PCT No.: PCT/AU2023/050452

§ 371 Date: Nov. 25, 2024

(30) Foreign Application Priority Data
May 27, 2022 (AU) 2022901434

(51) Int. Cl. G01N 33/68; C12Q 1/34; C12Q 1/527

(52) U.S. Cl. CPC G01N 33/6893; C12Q 1/34; C12Q 1/527; G01N 2333/4703; G01N 2333/4713; G01N 2333/8146; G01N 2333/918; G01N 2333/988; G01N 2400/40; G01N 2800/085; G01N 2800/52; G01N 2800/56

(57) ABSTRACT

The present disclosure relates generally to biomarkers of fibrosis. More particularly, the present disclosure relates to salivary biomarkers and their use in methods, apparatuses, compositions and kits for determining an indicator that is useful for assessing a likelihood of a presence, absence or degree of liver fibrosis in a human subject. In particular embodiments, the disclosed methods, apparatuses, compositions and kits are used to determine an indicator for assessing a likelihood of a presence, absence or development of liver cirrhosis in a subject.

### RELATED APPLICATIONS

This application claims priority to Australian Patent Application No. 2022901434 entitled “Biomarkers of fibrosis and uses therefor” filed 27 May 2022, the contents of which are incorporated herein by reference in their entirety.

### FIELD

This disclosure relates generally to biomarkers of fibrosis. More particularly, the present disclosure relates to salivary biomarkers and their use in methods, apparatuses, compositions and kits for determining an indicator that is useful for assessing a likelihood of a presence, absence or degree of liver fibrosis in a human subject. In particular embodiments, the disclosed methods, apparatuses, compositions and kits are used to determine an indicator for assessing a likelihood of a presence, absence or development of liver cirrhosis in a subject.

### BACKGROUND

Hepatic fibrosis is a common feature in the majority of chronic liver diseases (Roehlen et al., 2020. Cells 9(4):875), characterized by progressive substitution of liver parenchyma with scar tissue as a response to sustained injury (Kisseleva et al., 2021. Nat Rev Gastroenterol Hepatol 18(3):151-166). In its advanced stage, known as liver cirrhosis, it can cause serious complications such as ascites, bleeding from esophageal varices, hepatic encephalopathy, hepatocellular carcinoma (HCC) and liver failure (Bernardi et al., 2018. Nat Rev Gastroenterol Hepatol 15(12):753-764). Furthermore, the vast majority of HCC cases develop in the context of a cirrhotic liver, making it the third leading cause of cancer-related death worldwide (Sung et al., 2021. Global cancer statistics 2020: GLOBOCAN estimates of incidence and mortality worldwide for 36 cancers in 185 countries, CA: a cancer journal for clinicians 71(3):209-249). Thus, the reliable assessment of the degree of fibrosis is an important factor to guide therapeutic decisions and determine prognosis in patients with chronic liver disease (Friedrich-Rust et al., 2016. Nat Rev Gastroenterol Hepatol 13(7):402-411).

Liver biopsy is considered to be the gold standard for the assessment of liver fibrosis (Roehlen et al., 2020. supra). However, due to risks and other limitations, a biopsy is not suited for screening purposes (Heyens et al., 2021. Front Med 8:476). In addition, it cannot be implemented early at the onset of the disease, mostly reserved for high-risk patients and long-term follow-up (Vilar-Gomez et al., 2018. J Hepatol 68(2):305-315). For this reason, non-invasive methods to detect liver fibrosis have gained attention. Those methods consist of a “biological approach”, mainly using the quantification of biomarkers in body fluids, and a “physical approach”, using ultrasound- or magnetic resonance-based technologies (Castera et al., 2019. Gastroenterology 156(5):1264-1281. e4). In the biological approach, a score is calculated based on the measurement of a combination of clinical and laboratory variables (Francque et al., 2018. Acta Gastroenterol Belg 81(1):55-81). For instance, the Fibrosis-4 (FIB-4) score is composed of age, aspartate aminotransferase (AST), alanine aminotransferase (ALT) and platelet count, with a diagnostic accuracy of 80% (77% sensitivity and 79% specificity) (Xiao et al., 2017. Hepatology 66(5):1486-1501). Other scores use extracellular matrix-related molecules such as the Enhanced Liver Fibrosis (ELF) score, based on the measurement of type III procollagen peptide (PIIINP), hyaluronic acid (HA), and tissue inhibitor matrix metalloproteinase 1 (TIMP1) (Lichtinghagen et al., 2013. J Hepatol 59(2):236-42). Several studies report the ELF score as the most accurate serum-based test to detect advanced fibrosis and cirrhosis (AUC 0.78-0.84) (Friedrich-Rust et al., 2016. supra; Mayo et al., 2008. Hepatology 48(5):1549-1557).

### SUMMARY

The present disclosure is based in part on the finding that certain biomarkers in saliva are biomarkers for liver fibrosis. Notably, the levels of these salivary biomarkers were found to correlate with the degree or severity of liver fibrosis and to markedly increase in patients with cirrhosis of the liver, as compared to healthy individuals and patients with underlying liver disease. The present inventors have also found that the performance of these biomarkers for detecting and/or quantifying liver fibrosis and for screening and/or early diagnosis of liver cirrhosis can be improved through use of a diagnostic classifier (referred to herein as Saliva Liver Fibrosis (SALF) score). Based on these findings, methods, apparatuses, compositions and kits are disclosed, which take advantage of these biomarkers for determining a presence, absence or degree or severity of liver fibrosis, which can be used advantageously as an aid in diagnosis of a presence or risk of development of liver cirrhosis.

Accordingly, in one aspect, methods are disclosed for determining an indicator used in assessing a likelihood of a subject having a presence, absence or degree or severity of liver fibrosis. These methods generally comprise, consist or consist essentially of:
(1) determining a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of α-2-macroglobulin (A2MG), hyaluronic acid (HA), and tissue inhibitor matrix metalloproteinase 1 (TIMP1); and
(2) determining the indicator using the biomarker values.

Disclosed herein in another aspect are methods for determining an indicator used in assessing a likelihood that liver cirrhosis is present, absent or developing in a subject. These methods general comprise, consist or consist essentially of:
(1) determining a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of A2MG, HA, and TIMP1; and
(2) determining the indicator using the biomarker values.

In some embodiments, the indicator indicates a likelihood of a presence or development of liver cirrhosis if:
A2MG is present in the saliva sample at a higher level than in a reference saliva sample obtained from a healthy subject or from a subject with non-fibrotic liver disease;
HA is present in the saliva sample at a higher level than in a reference saliva sample obtained from a healthy subject or from a subject with non-fibrotic liver disease; and
TIMP1 is present in the saliva sample at a higher level than in a reference saliva sample obtained from a healthy subject or from a subject with non-fibrotic liver disease.

In some embodiments, the indicator indicates a likelihood of a presence or development of liver cirrhosis if:
A2MG is present in the saliva sample at a level corresponding to the level of A2MG in a reference saliva sample obtained from a subject with liver cirrhosis;
HA is present in the saliva sample at a level corresponding to the level of HA in a reference saliva sample obtained from a subject with liver cirrhosis; and
TIMP1 is present in the saliva sample at a level corresponding to the level of TIMP1 in a reference saliva sample obtained from a subject with liver cirrhosis.

In some embodiments, the indicator indicates a likelihood of the absence of liver cirrhosis if:
A2MG is present in the saliva sample at a lower level than in a reference saliva sample obtained from a subject with liver cirrhosis;
HA is present in the saliva sample at a lower level than in a reference saliva sample obtained from a subject with liver cirrhosis; and
TIMP1 is present in the saliva sample at a lower level than in a reference saliva sample obtained from a subject with liver cirrhosis.

In some embodiments, the indicator indicates a likelihood of the absence of liver cirrhosis if:
A2MG is present in the saliva sample at a level corresponding to the level of A2MG in a reference saliva sample obtained from a healthy subject or from a subject with non-fibrotic liver disease;
HA is present in the saliva sample at a level corresponding to the level of HA in a reference saliva sample obtained from a healthy subject or from a subject with non-fibrotic liver disease; and
TIMP1 is present in the saliva sample at a level corresponding to the level of TIMP1 in a reference saliva sample obtained from a healthy subject or from a subject with non-fibrotic liver disease.

Another aspect of the present disclosure provides methods for determining an indicator used in assessing a likelihood of a subject having a presence, absence or degree or severity of liver fibrosis. These methods generally comprise, consist or consist essentially of: (1) determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS); and
(2) determining the indicator using the biomarker value(s).

Disclosed herein in another aspect are methods for determining an indicator used in assessing a likelihood that liver cirrhosis is present, absent or developing in a subject. These methods general comprise, consist or consist essentially of:
(1) determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from A2MG, HA, TIMP1, CA1, KPNA3, and PGLS; and
(2) determining the indicator using the biomarker value(s).

In some embodiments, the at least one biomarker is represented by a biomarker signature selected from: [A2MG]; [HA]; [TIMP1]; [CA1]; [KPNA3]; [PGLS]; [A2MG:HA]; [A2MG:TIMP1]; [A2MG:CA1]; [A2MG:KPNA3]; [A2MG:PGLS]; [HA:TIMP1]; [HA:CA1]; [HA:KPNA3]; [HA:PGLS]; [TIMPi:CA1]; [TIMPi:KPNA3]; [TIMPi:PGLS]; [CA1:KPNA3]; [CA1:PGLS]; [KPNA3:PGLS]; [A2MG:HA:TIMP1]; [A2MG:HA:CA1]; [A2MG:HA:KPNA3]; [A2MG:HA:PGLS]; [A2MG:TIMP1:CA1]; [A2MG:TIMP1:KPNA3]; [A2MG:TIMP1:PGLS]; [A2MG:CA1:KPNA3]; [A2MG:CA1:PGLS]; [A2MG:KPNA3:PGLS]; [HA:TIMP1:CA1]; [HA:TIMP1:KPNA3]; [HA:TIMP1:PGLS]; [HA:CA1:KPNA3]; [HA:CA1:PGLS]; [HA:KPNA3:PGLS]; [TIMPi:CA1:KPNA3]; [TIMPi:CA1:PGLS]; [TIMPi:KPNA3:PGLS]; [CA1:KPNA3:PGLS]; [A2MG:HA:TIMP1:CA1]; [A2MG:HA:TIMP1:KPNA3]; [A2MG:HA:TIMP1:PGLS]; [A2MG:HA:CA1:KPNA3]; [A2MG:HA:CA1:PGLS]; [A2MG:HA:KPNA3:PGLS]; [A2MG:TIMP1:CA1:KPNA3]; [A2MG:TIMP1:CA1:PGLS]; [A2MG:TIMP1:KPNA3:PGLS]; [A2MG:CA1:KPNA3:PGLS]; [HA:TIMP1:CA1:KPNA3]; [HA:TIMP1:CA1:PGLS]; [HA:TIMP1:KPNA3:PGLS]; [HA:CA1:KPNA3:PGLS]; [TIMPi:CA1:KPNA3:PGLS]; [A2MG:HA:TIMP1:CA1:KPNA3]; [A2MG:HA:TIMP1:CA1:PGLS]; [A2MG:HA:TIMP1:KPNA3:PGLS]; [A2MG:HA:CA1:KPNA3:PGLS]; [A2MG:TIMP1:CA1:KPNA3:PGLS]; [HA:TIMP1:CA1:KPNA3:PGLS]; and [A2MG:HA:TIMP1:CA1:KPNA3:PGLS].

In some embodiments, a biomarker value is determined for 1, 2 or 3 biomarkers selected from A2MG, HA, and TIMP1 and the indicator is determined using the biomarker value(s). In some embodiments, a biomarker value is determined for 1, 2 or 3 biomarkers selected from CA1, KPNA3, and PGLS and the indicator is determined using the biomarker value(s). In some embodiments, biomarker values are determined for 2, 3, 4, 5 or 6 3 biomarkers selected from A2MG, HA, TIMP1, CA1, KPNA3, and PGLS and the indicator is determined using those biomarker values. In specific embodiments, biomarker values are determined for each of A2MG, HA and TIMP1 and the indicator is determined using those biomarker values. In other specific embodiments, biomarker values are determined for each of CA1, KPNA3, and PGLS and the indicator is determined using those biomarker values. In still other specific embodiments, biomarker values are determined for each of A2MG, HA, TIMP1, CA1, KPNA3, and PGLS and the indicator is determined using those biomarker values.

In some embodiments, the indicator indicates a likelihood of a presence or development of liver fibrosis or liver cirrhosis if:
A2MG is present in the saliva sample at a higher level than in a reference saliva sample obtained from a healthy subject or from a subject with non-fibrotic liver disease;
HA is present in the saliva sample at a higher level than in a reference saliva sample obtained from a healthy subject or from a subject with non-fibrotic liver disease;
TIMP1 is present in the saliva sample at a higher level than in a reference saliva sample obtained from a healthy subject or from a subject with non-fibrotic liver disease;
CA1 is present in the saliva sample at a higher level than in a reference saliva sample obtained from a healthy subject or from a subject with non-fibrotic liver disease;
KPNA3 is present in the saliva sample at a lower level than in a reference saliva sample obtained from a healthy subject or from a subject with non-fibrotic liver disease; and/or
PGLS is present in the saliva sample at a higher level than in a reference saliva sample obtained from a healthy subject or from a subject with non-fibrotic liver disease.

In some embodiments, the indicator indicates a likelihood of a presence or development of liver fibrosis or liver cirrhosis if:
A2MG is present in the saliva sample at a level corresponding to the level of A2MG in a reference saliva sample obtained from a subject with liver fibrosis or liver cirrhosis;
HA is present in the saliva sample at a level corresponding to the level of HA in a reference saliva sample obtained from a subject with liver fibrosis or liver cirrhosis;
TIMP1 is present in the saliva sample at a level corresponding to the level of TIMP1 in a reference saliva sample obtained from a subject with liver fibrosis or liver cirrhosis;
CA1 is present in the saliva sample at a level corresponding to the level of CA1 in a reference saliva sample obtained from a subject with liver fibrosis or liver cirrhosis;
KPNA3 is present in the saliva sample at a level corresponding to the level of KPNA3 in a reference saliva sample obtained from a subject with liver fibrosis or liver cirrhosis; and/or PGLS is present in the saliva sample a level corresponding to the level of PGLS in a reference saliva sample obtained from a subject with liver fibrosis or liver cirrhosis.

In some embodiments, the indicator indicates a likelihood of the absence of liver fibrosis or liver cirrhosis if:
A2MG is present in the saliva sample at a lower level than in a reference saliva sample obtained from a subject with liver fibrosis or liver cirrhosis;
HA is present in the saliva sample at a lower level than in a reference saliva sample obtained from a subject with liver fibrosis or liver cirrhosis;
TIMP1 is present in the saliva sample at a lower level than in a reference saliva sample obtained from a subject with liver fibrosis or liver cirrhosis;
CA1 is present in the saliva sample at a lower level than in a reference saliva sample obtained from a subject with liver fibrosis or liver cirrhosis;
KPNA3 is present in the saliva sample at a higher level than in a reference saliva sample obtained from a subject with liver fibrosis or liver cirrhosis; and/or PGLS is present in the saliva sample at a lower level than in a reference saliva sample obtained from a subject with liver fibrosis or liver cirrhosis.

In some embodiments, the indicator indicates a likelihood of the absence of liver fibrosis or liver cirrhosis if:
A2MG is present in the saliva sample at a level corresponding to the level of A2MG in a reference saliva sample obtained from a healthy subject or from a subject with non-fibrotic liver disease;
HA is present in the saliva sample at a level corresponding to the level of HA in a reference saliva sample obtained from a healthy subject or from a subject with non-fibrotic liver disease;
TIMP1 is present in the saliva sample at a level corresponding to the level of TIMP1 in a reference saliva sample obtained from a healthy subject or from a subject with non-fibrotic liver disease
CA1 is present in the saliva sample at a level corresponding to the level of CA1 in a reference saliva sample obtained from a healthy subject or from a subject with non-fibrotic liver disease;
KPNA3 is present in the saliva sample at a level corresponding to the level of KPNA3 in a reference saliva sample obtained from a healthy subject or from a subject with non-fibrotic liver disease; and/or
PGLS is present in the saliva sample at a level corresponding to the level of PGLS in a reference saliva sample obtained from a healthy subject or from a subject with non-fibrotic liver disease.

The methods may further comprise applying a function to biomarker values to yield at least one functionalized biomarker value and determining the indicator using the at least one functionalized biomarker value. In representative examples, the function includes at least one of: (a) multiplying biomarker values; (b) dividing biomarker values; (c) adding biomarker values; (d) subtracting biomarker values; (e) a weighted sum of biomarker values; (f) a log sum of biomarker values; (g) a geometric mean of biomarker values; (h) a sigmoidal function of biomarker values; and (i) normalization of biomarker values.

In some embodiments, the methods further comprise combining the biomarker values, optionally with clinical parameters, to provide a composite score and determining the indicator using the composite score. In non-limiting examples of this type, the biomarker values are combined by adding, multiplying, subtracting, and/or dividing biomarker values.

In some embodiments, the methods further comprise analyzing the biomarker value(s), functionalized biomarker value(s) or composite score with reference to a corresponding reference biomarker value range or cut-off values, functionalized biomarker value range or cut-off values, or reference composite score range or cut-off values, to determine the indicator.

Suitably, the indicator indicates a likelihood of a presence or degree or severity of liver fibrosis if the biomarker value(s) or composite score is indicative of the levels of the biomarkers in the sample that correlate with an increased likelihood of a presence or degree or severity of liver fibrosis relative to a predetermined reference biomarker value range or cut-off value. In some embodiments, the indicator indicates a likelihood of a presence of liver cirrhosis if the biomarker value(s) or composite score is indicative of the levels of the biomarkers in the sample that correlate with an increased likelihood of a presence of liver cirrhosis relative to a predetermined reference biomarker value range or cut-off value.

In any of the aspects and embodiments disclosed herein, individual biomarker values suitably represent a measured amount, abundance or concentration of a corresponding biomarker in the sample.

In any of the aspects and embodiments disclosed herein, the subject is suitably a mammalian subject such as a human subject.

In any of the aspects disclosed herein, the subject may be asymptomatic or may have at least one clinical sign of liver fibrosis or liver cirrhosis.

In any of the aspects and embodiments disclosed herein, the subject has a disease selected from hepatitis (e.g., a viral hepatitis such as Hepatitis A, Hepatitis B, Hepatitis C, Hepatitis D and Hepatitis E, or an autoimmune hepatitis), fatty liver disease (e.g., non-alcoholic fatty liver disease (NAFLD) (also referred to as metabolic associated fatty liver disease (MAFLD)), non-alcoholic steatohepatitis (NASH), alcoholic fatty liver disease (AFLD) and alcoholic steatohepatitis (ASH)), alcoholic liver disease (ALD), primary sclerosing cholangitis (PSC), and primary biliary cholangitis (PBC), hemochromatosis, Wilson's disease, drug-induced liver disease, liver cancer (e.g., hepatocellular carcinoma), pediatric liver diseases that cause fibrosis and cirrhosis and all other recognized causes of liver fibrosis and cirrhosis.

In a related aspect, methods are disclosed for monitoring liver fibrosis status or treatment of a subject. These methods generally comprise, consist or consist essentially of:
(1) determining a biomarker value for each of a plurality of biomarkers in a first saliva sample obtained from the subject, wherein a respective biomarker value is indicative of a level of a corresponding biomarker in the sample, and wherein the plurality of biomarkers comprises, consists or consists essentially of A2MG, HA, and TIMP1;
(2) determining a first indicator using the biomarker values;
(3) determining a biomarker value for each of the plurality of biomarkers in a second saliva sample obtained from the subject, wherein a respective biomarker value is indicative of a level of a corresponding biomarker in the second sample;
(4) determining a second indicator using the biomarker values; and
(5) comparing the first indicator with the second indicator, thereby monitoring the liver fibrosis status or treatment of the subject.

In another related aspect, methods are disclosed for monitoring liver fibrosis status or treatment of a subject. These methods generally comprise, consist or consist essentially of:
(1) determining a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from A2MG, HA, TIMP1, CA1, KPNA3 and PGLS;
(2) determining a first indicator using the biomarker value(s);
(3) determining a biomarker value for each of the at least one biomarkers, for which biomarker values were determined in the first saliva sample, in a second saliva sample obtained from the subject, wherein a respective biomarker value is indicative of a level of a corresponding biomarker in the second sample;
(4) determining a second indicator using the biomarker value(s); and
(5) comparing the first indicator with the second indicator, thereby monitoring the liver fibrosis status or treatment of the subject.

The second indicator may indicate reduced liver fibrosis relative to the liver fibrosis indicated by the first indicator, which is indicative of improved liver fibrosis status or effective treatment of the subject. Alternatively, the second indicator may indicate unchanged liver fibrosis relative to the liver fibrosis indicated by the first indicator, which is indicative of an unchanged liver fibrosis status or a treatment that is effective in slowing progression of disease of the subject. In other embodiments, the second indicator indicates increased liver fibrosis relative to the liver fibrosis indicated by the first indicator, which is indicative of worsening liver fibrosis status or an ineffective treatment of the subject.

In some embodiments, the first sample is obtained from the subject before undergoing a therapeutic regimen for treating liver fibrosis and the second sample is obtained from the subject after undergoing the therapeutic regimen.

In still another aspect, apparatuses are disclosed for determining an indicator used in assessing a likelihood of a subject having a presence, absence or degree or severity of liver fibrosis. These apparatuses general comprise, consist or consist essentially of at least one electronic processing device that:
determines a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of A2MG, HA, and TIMP1; and
determines the indicator using the biomarker values.

In a related aspect, apparatuses are disclosed for determining an indicator used in assessing a likelihood that liver cirrhosis is present, absent or developing in a subject. These apparatuses general comprise, consist or consist essentially of at least one electronic processing device that:
determines a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of A2MG, HA, and TIMP1; and
determines the indicator using the biomarker values.

Another aspect of the present disclosure provides apparatuses for determining an indicator used in assessing a likelihood of a subject having a presence, absence or degree or severity of liver fibrosis. These apparatuses general comprise, consist or consist essentially of at least one electronic processing device that:
determines a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from A2MG, HA, TIMP1, CA1, KPNA3 and PGLS; and
determines the indicator using the biomarker value(s).

In a related aspect, apparatuses are disclosed for determining an indicator used in assessing a likelihood that liver cirrhosis is present, absent or developing in a subject. These apparatuses general comprise, consist or consist essentially of at least one electronic processing device that:
determines a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from A2MG, HA, TIMP1, CA1, KPNA3 and PGLS; and
determines the indicator using the biomarker value(s).

Another aspect of the present disclosure provides compositions, suitably for determining an indicator used in assessing a likelihood of a subject having a presence, absence or degree or severity of liver fibrosis, or a likelihood that liver cirrhosis is present, absent or developing in a subject. These compositions generally comprise, consist or consist essentially of a mixture of a saliva sample obtained from the subject, and for each of a plurality of biomarkers an antibody or antigen-binding fragment that binds specifically to the biomarker, wherein the plurality of biomarkers comprises, consists or consists essentially of A2MG, HA, and TIMP1.

In another compositions are disclosed, suitably for determining an indicator used in assessing a likelihood of a subject having a presence, absence or degree or severity of liver fibrosis, or a likelihood that liver cirrhosis is present, absent or developing in a subject. These compositions generally comprise, consist or consist essentially of a mixture of a saliva sample obtained from the subject, and for at least one (e.g., 1, 2, 3, 4, 5, 6, etc.) biomarker an antibody or antigen-binding fragment that binds specifically to the biomarker, wherein the at least one biomarker is selected from A2MG, HA, TIMP1, CA1, KPNA3 and PGLS.

Individual antibodies or antigen-binding fragments may be labeled. In some embodiments, the composition comprises a plurality of antibodies or antigen-binding fragments, each of which specifically binds to a different biomarker and is associated with the same label or a different label, as compared to the biomarker specificity and label associated with other antibodies or antigen-binding fragments of the composition. In illustrative examples of this type, the labels associated with different antibodies or antigen-binding fragments are detectably distinct.

In a further aspect, methods are disclosed for inhibiting the development or progression of liver fibrosis in a subject. These methods generally comprise, consist or consist essentially of exposing the subject to a treatment regimen for treating liver fibrosis at least in part on the basis that the subject is determined by the indicator-determining method as broadly described above and elsewhere herein as having a likelihood of a presence or degree or severity of liver fibrosis.

In a related aspect, methods are disclosed for inhibiting the development or progression of liver cirrhosis in a subject. These methods generally comprise, consist or consist essentially of exposing the subject to a treatment regimen for treating liver cirrhosis at least in part on the basis that the subject is determined by the indicator-determining method as broadly described above and elsewhere herein as having a likelihood of a presence of liver cirrhosis.

In some embodiments, the subject has been administered a treatment regimen prior to undertaking the indicator-determining method. In other embodiments, the subject has not undergone a treatment regimen prior to undertaking the indicator-determining method.

In some embodiments, the treatment methods further comprise: taking a sample from the subject and determining an indicator indicative of a likelihood of a presence or degree or severity of liver fibrosis or of a presence of liver cirrhosis using the indicator-determining method. In some of the same or other embodiments, the methods further comprise: sending a sample obtained from the subject to a laboratory at which the indicator is determined according to the indicator-determining method, and optionally receiving the indicator from the laboratory.

A further aspect of the present disclosure provides kits for determining an indicator used in assessing a likelihood of a subject having a presence, absence or degree or severity of liver fibrosis, or a likelihood that liver cirrhosis is present, absent or developing in a subject. These kits generally comprise for each of a plurality of biomarkers an antibody or antigen-binding fragment that binds specifically to the biomarker, wherein the plurality of biomarkers comprises, consists or consists essentially of A2MG, HA, and TIMP1.

In another aspect, kits are disclosed for determining an indicator used in assessing a likelihood of a subject having a presence, absence or degree or severity of liver fibrosis, or a likelihood that liver cirrhosis is present, absent or developing in a subject. These kits generally comprise for at least one biomarker an antibody or antigen-binding fragment that binds specifically to the biomarker, wherein the at least one biomarker is selected from A2MG, HA, TIMP1, CA1, KPNA3 and PGLS.

The kits may further comprise any one or more of: at least one reagent for preparing a saliva sample for biomarker analysis; buffer(s), positive and negative controls, and reaction vessel(s). Suitably, the kits may further comprise instructions for performing the indicator-determining methods as broadly described above and elsewhere herein.

### BRIEF DESCRIPTION OF THE DRAWINGS

FIG. 1 is a schematic representation illustrating the development of the Saliva Liver Fibrosis (SALF) score. HA—hyaluronic acid; TIMP-1-tissue inhibitor of metalloproteinase 1; A2MG—α-2-macroglobulin; P3NP—procollagen III amino-terminal propeptide; GGT—γ-glutamyl transferase; TE—transient elastography; LSM—liver stiffness measurement; HC—healthy controls; NF—non-fibrotic liver disease; IF—intermediate degree of fibrosis; LC—liver cirrhosis.

FIG. 2 is a graphical representation showing concentrations of HA, P3NP, TIMP-1, A2MG, total bilirubin and GGT in paired serum (left) and saliva (right) in the training set: healthy controls (HC), patients with liver disease without fibrosis (NF), patients with intermediate degrees of fibrosis (IF) or cirrhosis (LC). Significant differences are indicated by *(p<0.05), **(p<0.01), ***(p<0.001) and ****(p<0.0001).

FIG. 3 is a graphical representation showing ROC analysis of various biomarker panels for distinguishing between liver cirrhosis patients and healthy controls. In particular, ROC curves are shown for the following panels: HA+TIMP1+A2MG, TIMP1+HA+GGT, HA+GGT+A2MG, HA+P3NP+GGT, Bilirubin+TIMP1+GGT, A2MG+GGT+Bilirubin and Bilirubin+P3NP+GGT.

FIG. 4 is a graphical and tabular representation showing ROC analysis of the Saliva Liver Fibrosis (SALF) score. (A) The SALF score for each individual was calculated using a logistic regression model combining the measurement of HA, TIMP-1 and A2MG (cut-off of 0.55 indicated as a dashed diagonal line). (B) The performance of the SALF score was compared with its individual components, (C) between serum and saliva samples, and (D and E) with other serum-based diagnostic models used for the detection of liver cirrhosis. SALF: Saliva Liver Fibrosis score; ELF: Enhanced Liver Fibrosis score; FIB-4: fibrosis-4 score; APRI: AST-to-platelet ratio index; PPV:

### Positive Predictive Value; NPV: Negative Predictive Value

FIG. 5 is a graphical representation showing validation of the SALF score in an independent cohort. (A) The concentrations of HA, TIMP-1 and A2MG were measured in the saliva of HC, NF, IF and LC patients. (B) The SALF score was calculated and (C) ROC analysis was performed to assess the performance of the SALF score for the diagnosis of liver fibrosis (LC+IF vs HC+NF) compared to the individual components.

FIG. 6 is a graphical representation showing quantification of HA, TIMP-1 and A2MG in the saliva of healthy controls, patients with liver disease without fibrosis, liver fibrosis and cirrhosis patients in the (A) training and (B) validation cohorts.

FIG. 7 is a graphical representation showing quantification of CA1, PGLS and KPNA3 in the saliva of healthy controls, patients with liver disease and liver cirrhosis. The graphs represent the integrated normalized protein intensity. * p<0.01, ** p<0.01 and *** p<0.001.

FIG. 8 is a photographic representation showing: (A) Confirmation of the proteins using western blot analysis of the salivary proteins of healthy, liver disease and liver cirrhosis patients. (B) Immunohistochemistry staining for CA1, PGLS and KPNA3 in liver sections from Mdr2 knockout mouse (fibrosis stage F3/F4) and wild-type animals.

FIG. 9 is a graphical representation showing: (A) Concentration of CA1, PGLS and KPNA3 in serum and (B) Correlation between serum and salivary concentrations.

Some figures and text contain color representations or entities. Color illustrations are available from the Applicant upon request or from an appropriate Patent Office. A fee may be imposed if obtained from a Patent Office.

### DETAILED DESCRIPTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

The term “about” as used herein refers to the usual error range for the respective value readily known to the skilled person in this technical field. Reference to “about” in connection with a value or parameter herein includes (and describes) embodiments that are directed to that value or parameter per se. In specific embodiments, the term “about” refers to a value or parameter (e.g., quantity, level, concentration, number, frequency, percentage, dimension, size, amount, weight or length) that varies by as much 15, 14, 13, 12, 11, 10, 9, 8, 7, 6, 5, 4, 3, 2 or 1% to a reference value or parameter.

The term “aiding diagnosis” is used herein to refer to methods that assist in making a clinical determination regarding the presence, or nature, of a particular type of symptom or condition of a disease or disorder (e.g., liver fibrosis, liver cirrhosis, etc.). For example, a method of aiding diagnosis of a disease or condition as disclosed for example herein can comprise measuring certain biomarkers (e.g., the biomarkers disclosed herein) in a biological sample (e.g., saliva) of an individual.

The “amount”, “level” or “abundance” of a biomarker is a detectable level, amount or abundance in a sample. These can be measured by methods known to one skilled in the art and also disclosed herein. These terms encompass a quantitative amount, abundance or level (e.g., weight or moles), a semi-quantitative amount, abundance or level, a relative amount, abundance or level (e.g., weight % or mole % within class), a concentration, and the like. Thus, these terms encompass absolute or relative amounts, abundances or levels or concentrations of a biomarker in a sample.

As used herein, “and/or” refers to and encompasses any and all possible combinations of one or more of the associated listed items, as well as the lack of combinations when interpreted in the alternative (or).

The term “antibody”, as used herein, means any antigen-binding molecule or molecular complex comprising at least one complementarity determining region (CDR) that binds specifically to or interacts with a particular antigen (e.g., A2MG, HA or TIMP1). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or V_{H}) and a heavy chain constant region. The heavy chain constant region comprises three domains, C_{H1}, C_{H2} and C_{H3}. Each light chain comprises a light chain variable region (which may be abbreviated as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of an antibody of the invention (or antigen-binding portion thereof) may be identical to the human germline sequences, or may be naturally or artificially modified. An amino acid consensus sequence may be defined based on a side-by-side analysis of two or more CDRs. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides, glycosaminoglycans), phospholipids, and proteins.

The terms “antigen-binding fragment”, “antigen-binding portion”, “antigen-binding domain” and “antigen-binding site” are used interchangeably herein to refer to a part of an antigen-binding molecule that participates in antigen-binding. These terms include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present disclosure include antibodies and their antigen-binding fragments. The term “antigen-binding molecule” includes antibodies and antigen-binding fragments of antibodies.

As used herein, the term “array” refers to an arrangement of capture reagents on a substrate, in which individual capture reagents bind specifically to a particular molecule (e.g., protein or antigen). In preferred embodiments, the capture reagents are antibodies or antigen-binding fragments.

As used herein, the term “biomarker” refers to a naturally occurring biological molecule present in a subject at varying concentrations useful in in assessing a likelihood of a subject having a presence, absence or degree of a disease or condition. For example, the biomarker can be a protein or polysaccharide (e.g., glycosaminoglycan) present in higher or lower amounts in saliva a subject. In certain embodiments, the biomarker is a protein selected from A2MG, TIMP1, CA1, KPNA3 and PGLS or the glycosaminoglycan, HA.

The term “biomarker value” refers to a value measured or functionalized for at least one corresponding biomarker of a subject and which is typically indicative of an abundance or concentration of a biomarker in a sample obtained from the subject. Thus, the biomarker values could be measured biomarker values, which are values of biomarkers measured for the subject. These values may be quantitative or qualitative. For example, a measured biomarker value may refer to a presence or absence of a biomarker or may refer to an amount, level or abundance of a biomarker in a sample. The measured biomarker values can be values relating to raw or normalized biomarker levels (e.g., a raw, non-normalized biomarker level, or a normalized biomarker levels that is determined relative to an internal or external control biomarker level) and to mathematically transformed biomarker levels. Alternatively, the biomarker values could be functionalized biomarker values, which are values that have been functionalized from one or more measured biomarker values, for example by applying a function to the one or more measured biomarker values. Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies and in representative examples, the biomarker values relate to a level of activity or abundance of an expression product or other measurable molecule, quantified using a nucleic acid assay such as real-time polymerase chain reaction (RT-PCR), sequencing or the like.

The terms “biomarker signature”, “signature”, “biomarker panel”, “panel” and the like are used interchangeably herein and refer to one or a combination of biomarkers whose expression is an indicator, e.g., predictive, diagnostic, and/or prognostic. A biomarker signature may comprise at least 1, 2, 3, 4, 5, 6, 7, 8, 9, 10, 11, 12, 13, 14, 15, 16, or more biomarkers. A biomarker signature can further comprise one or more controls or internal standards. In certain embodiments, a biomarker signature comprises at least one biomarker, or indication thereof, that serves as an internal standard. In other embodiments, a biomarker signature comprises an indication of one or more types of biomarkers. The term “indication” as used herein in this context merely refers to a situation where the biomarker signature contains symbols, data, abbreviations or other similar indicia for a biomarker, rather than the biomarker molecular entity itself. The term “biomarker signature” is also used herein to refer to a biomarker value or combination of at least two biomarker values, wherein individual biomarker values correspond to values of biomarkers that can be measured or functionalized from one or more subjects, which combination is characteristic of a discrete condition, stage of condition, subtype of condition or a prognosis for a discrete condition, stage of condition, subtype of condition. The term “signature biomarkers” is used to refer to a subset of the biomarkers that have been identified for use in a biomarker signature that can be used in performing a clinical assessment, such as to rule in or rule out a specific condition, different stages or severity of conditions, subtypes of different conditions or different prognoses. The number of signature biomarkers will vary, but is typically of the order of 16 or less (e.g., 16, 15, 14, 13, 12, 11, 10, 9, 8, 7, 6, 5, 4, 3, 2 or 1). In preferred embodiments, the biomarker signature comprises, consists or consists essentially of 1, 2, 3, 4, 5 or 6 biomarkers.

As use herein, the term “binds”, “specifically binds to” or is “specific for” refers to measurable and reproducible interactions such as binding between a target and an antibody, which is determinative of a presence of the target in a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by ELISA or radioimmunoassay (RIA). In certain embodiments, an antibody that specifically binds to a target has a dissociation constant (Kd) of 1 μM, 100 nM, 10 nM, 1 nM, or 0.1 nM. In certain embodiments, an antibody specifically binds to an epitope on a protein or polysaccharide (e.g., a glycosaminoglycan) that is conserved among the proteins or polysaccharides from different species. In another embodiment, specific binding can include, but does not require exclusive binding.

The term “clinical parameter”, as used herein, refers any clinical measure of a health or disease status of a subject, such as, without limitation, age, ethnicity, gender, Hepatitis virus (e.g., Hepatitis B) antigen, Hepatitis virus (e.g., Hepatitis B) nucleic acid, alanine aminotransferase (ALT}level, alkaline phosphatase(ALP) level, platelet count, standard deviation of red blood cell distribution width (RDW-SD), albumin level, bilirubin level, γ-glutamyl transpeptidase (GGT) level and α-fetoprotein (AFP) level.

As used herein, the term “clinical sign”, or simply “sign”, refers to objective evidence of a presence of disease or condition (e.g., liver fibrosis, liver cirrhosis, etc.) in a subject. Symptoms and/or signs associated with a particular disease or condition and the evaluation of such signs are routine and known in the art. Examples of symptoms of liver fibrosis include poor appetite, feeling weak, unexplained exhaustion, unexplained weight loss, nausea and vomiting and discomfort or mild pain in upper right abdomen. Non-limiting examples of symptoms and signs of liver cirrhosis include a tendency to bruise or bleed easily, edema or fluid retention in the lower legs, ankles or feet, jaundice, ascites or abdominal bloating from a buildup of fluid, itchy skin, increased sensitivity to medications and their side effects, problems with certain cognitive functions, such as memory, concentration or sleeping and darkening of urine.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “composite score” refers to an aggregation of the obtained values for biomarkers measured in a sample from a subject, optionally in combination with one or more patient clinical parameters or signs. In some embodiments, the obtained biomarker values are normalized to provide a composite score for each subject tested. When used in the context of a risk categorization table and correlated to a stratified population grouping or cohort population grouping based on a range of composite scores in a risk categorization table, the “biomarker composite score” may be used, at least in part, by a machine learning system to determine the “risk score” for each subject tested wherein the numerical value (e.g., a multiplier, a percentage, etc.) indicating increased likelihood of having a presence, absence or degree of a disclosed condition (e.g., liver fibrosis or liver cirrhosis) for the stratified grouping becomes the “risk score”.

As used herein, the term “correlates” or “correlates with” and like terms, refers to a statistical association between two or more things, such as events, characteristics, outcomes, numbers, data sets, etc., which may be referred to as “variables”. It will be understood that the things may be of different types. Often the variables are expressed as numbers (e.g., measurements, values, likelihood, risk), wherein a positive correlation means that as one variable increases, the other also increases, and a negative correlation (also called anti-correlation) means that as one variable increases, the other variable decreases. In various embodiments, correlating a biomarker or biomarker signature with a presence or absence of a condition (e.g., a condition selected from a healthy condition, a non-fibrotic condition (e.g., non-fibrotic liver disease), a high degree of liver fibrosis correlating with presence of liver cirrhosis, a degree of liver fibrosis (also referred to herein as an “intermediate degree of liver fibrosis”) intermediate a healthy or non-fibrotic condition and a high degree of liver fibrosis correlating with presence of liver cirrhosis, etc.), comprises determining a presence, absence, level or amount of a plurality of biomarkers in a subject that has that condition; or in persons known to be free of that condition. In specific embodiments, a profile of biomarker levels, absences or presences is correlated to a global probability or a particular outcome, using receiver operating characteristic (ROC) curves.

The term “cut-off value” as used herein is an abundance, level or amount (or concentration) which may be an absolute level or a relative abundance, level or amount (or concentration), which is indicative of whether a subject has a particular disease or condition (e.g., a healthy condition, a non-fibrotic condition (e.g., non-fibrotic liver disease), an intermediate degree of liver fibrosis, and a high degree of liver fibrosis correlating with presence of liver cirrhosis, etc.). Depending on the biomarker or combination of biomarkers, a subject is regarded as having the disease or condition, or being at risk of having the disease or condition, if either the level of the biomarker(s) detected and determined, respectively, is lower than the cut-off value, or the level of the biomarker(s) detected and determined, respectively, is higher than the cut-off value.

As used herein, the terms “detectably distinct” and “detectably different” are used interchangeably to refer to a signal that is distinguishable or separable by a physical property either by observation or by instrumentation. For example, a fluorophore is readily distinguishable either by spectral characteristics or by fluorescence intensity, lifetime, polarization or photo-bleaching rate from another fluorophore in a sample, as well as from additional materials that are optionally present. In certain embodiments, the terms “detectably distinct” and “detectably different” refer to a set of labels (such as dyes, suitably organic dyes) that can be detected and distinguished simultaneously.

As used herein, the phrase “developing a classifier” refers to using input variables to generate an algorithm or classifier capable of distinguishing between two or more states (e.g., a condition selected from a healthy condition, a non-fibrotic condition (e.g., non-fibrotic liver disease), a high degree of liver fibrosis correlating with presence of liver cirrhosis, an intermediate degree of liver fibrosis, and a high degree of liver fibrosis correlating with presence of liver cirrhosis).

As used herein, the terms “diagnosis”, “diagnosing” and the like are used interchangeably herein to encompass determining the likelihood that a subject will develop a condition, or the existence or nature of a condition in a subject. These terms also encompass determining the severity of disease or episode of disease, as well as in the context of rational therapy, in which the diagnosis guides therapy, including initial selection of therapy, modification of therapy (e.g., adjustment of dose or dosage regimen), and the like. By “likelihood” is meant a measure of whether a subject with particular measured or functionalized biomarker values actually has a condition (or not), suitably based on a given mathematical model. An increased likelihood for example may be relative or absolute and may be expressed qualitatively or quantitatively. For instance, an increased likelihood may be determined simply by determining the subject's measured biomarker values for at least 1, 2, 3, 4, 5, 6, 7, 8, 9 or 10 biomarkers (e.g., 1, 2, 3, 4, 5 or 6 biomarkers) and placing the subject in an “increased likelihood” category, based upon previous population studies. The term “likelihood” is also used interchangeably herein with the term “probability”. The term “risk” relates to the possibility or probability of a particular event occurring at some point in the future. “Risk stratification” refers to an arraying of known clinical risk factors to allow physicians to classify patients into a low, moderate, high or highest risk of having or developing a particular disease or condition.

The term “differentially expressed” refers to differences in the quantity and/or the frequency of a biomarker present in a sample obtained from patients having, for example, a first condition (e.g., a healthy or non-fibrotic liver disease) as compared to subjects with a second condition (e.g., liver fibrosis or liver cirrhosis). For example, a biomarker can be a polypeptide which is present at an elevated level or at a decreased level in samples of patients with liver cirrhosis compared to samples of healthy subjects or subjects with non-fibrotic liver disease. A biomarker can be differentially present in terms of quantity, frequency or both.

The term “discrimination performance” refers to numeric representation of the index including, for example, sensitivity, specificity, positive predictability, negative predictability or accuracy. The term “discrimination performance” may also refer to a value computed by the functions of the indexes. For example, sensitivity, specificity, positive predictive value, negative predictive value and accuracy may each be used as the discrimination performance, or alternatively, the sum of two or more indexes, e.g., the sum of sensitivity and specificity, the sum of sensitivity and positive predictive value, or the sum of negative predictive value and accuracy, may be used as the discrimination performance.

“Fluorophore” as used herein to refer to a moiety that absorbs light energy at a defined excitation wavelength and emits light energy at a different defined wavelength. Examples of fluorescence labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, 25 Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red and Texas Red-X.

As used herein, the term “higher” with reference to a biomarker measurement refers to a statistically significant and measurable difference in the level of a biomarker compared to the level of another biomarker or to a control level where the biomarker measurement is greater than the level of the other biomarker or the control level. The difference is suitably at least about 10%, or at least about 20%, or of at least about 30%, or of at least about 40%, or at least about 50%.

As used herein, the term “increase” or “increased” with reference to a biomarker level refers to a statistically significant and measurable increase in the biomarker level compared to the level of another biomarker or to a control level. The increase is suitably an increase of at least about 10%, or an increase of at least about 20%, or an increase of at least about 30%, or an increase of at least about 40%, or an increase of at least about 50%.

The term “indicator” as used herein refers to a result or representation of a result, including any information, number (e.g., biomarker value including functionalized biomarker value and composite score), ratio, signal, sign, mark, or note by which a skilled artisan can estimate and/or determine a likelihood or risk of whether or not a subject is suffering from a given disease or condition. In the case of the present disclosure, the “indicator” may optionally be used together with other clinical characteristics, to arrive at a diagnosis (that is, the occurrence or nonoccurrence) of a condition disclosed herein in a subject. That such an indicator is “determined” is not meant to imply that the indicator is 100% accurate. The skilled clinician may use the indicator together with other clinical parameters or signs to arrive at a diagnosis.

As used herein, “instructional material” includes a publication, a recording, a diagram, or any other medium of expression which can be used to communicate the usefulness of the compositions and methods of the disclosure. The instructional material of the kit of the disclosure may, for example, be affixed to a container which contains the therapeutic or diagnostic agents of the disclosure or be shipped together with a container which contains the therapeutic or diagnostic and/or prognostic agents of the disclosure.

The term “label” is used herein in a broad sense to refer to an agent that is capable of providing a detectable signal, either directly or through interaction with one or more additional members of a signal producing system and that has been artificially added, linked or attached via chemical manipulation to a molecule. Labels can be visual, optical, photonic, electronic, acoustic, optoacoustic, by mass, electro-chemical, electro-optical, spectrometry, enzymatic, or otherwise chemically, biochemically hydrodynamically, electrically or physically detectable. Labels can be, for example tailed reporter, marker or adapter molecules. In specific embodiments, a molecule such as a nucleic acid molecule is labeled with a detectable molecule selected form the group consisting of radioisotopes, fluorescent compounds, bioluminescent compounds, chemiluminescent compounds, metal chelators or enzymes. Examples of labels include, but are not limited to, the following radioisotopes (e.g., ³H, ¹⁴C, ³⁵s, ¹²⁵I, ¹³¹I), fluorescent labels (e.g., FITC, rhodamine, lanthanide phosphors), luminescent labels such as luminol; enzymatic labels (e.g., horseradish peroxidase, R-galactosidase, luciferase, alkaline phosphatase, acetylcholinesterase), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags).

As used herein, the term “liver fibrosis” or “fibrosis of the liver” refers to an excessive accumulation in the liver of extracellular matrix proteins, which could include collagens (I, III, and IV), fibronectin, undulin, elastin, laminin, hyaluronan, and proteoglycans resulting from inflammation and liver cell death. Liver fibrosis, if left untreated, may progress to cirrhosis, liver failure, or liver cancer. Cirrhosis, the end-stage of progressive liver fibrosis, is characterized by septum formation and rings of scar that surround nodules of hepatocytes. Typically, fibrosis requires years or decades to become clinically apparent, but notable exceptions in which cirrhosis develops over months may include pediatric liver disease (e.g., biliary atresia), drug-induced liver disease, and viral hepatitis associated with immunosuppression after liver transplantation.

As used herein, the term “lower” with reference to a biomarker measurement refers to a statistically significant and measurable difference in the level of a biomarker compared to the level of another biomarker or to a control level where the biomarker measurement is less than the level of the other biomarker or the control level. The difference is suitably at least about 10%, or at least about 20%, or of at least about 30%, or of at least about 40%, or at least about 50%.

As used herein, the term “normalization” and its derivatives, when used in conjunction with measurement of biomarkers across samples and time, refer to mathematical methods, including but not limited to multiple of the median (MoM), standard deviation normalization, sigmoidal normalization, etc., where the intention is that these normalized values allow the comparison of corresponding normalized values from different datasets in a way that eliminates or minimizes differences and gross influences.

As used herein, the term “obtained” refers to come into possession. Samples so obtained include, for example, protein and/or polysaccharide (e.g., comprising glycosaminoglycan) extracts isolated or derived from a particular source (e.g., saliva).

As used herein, the term “panel” refers to specific combination of biomarkers used to determine an indicator for assessing a likelihood that a condition as disclosed herein is present, absent or developing in a subject. The term “panel” may also refer to an assay comprising a set of biomarkers used for such a determination. This term can also refer to a profile or index of expression patterns of one or more biomarkers described herein. The number of biomarkers useful for a biomarker panel is based on the sensitivity and specificity value for the particular combination of biomarker values.

“Protein”, “polypeptide” and “peptide” are used interchangeably herein to refer to a polymer of amino acid residues and to variants or synthetic analogues of the same.

As used herein, the term “reduce” or “reduced” with reference to a biomarker level refers to a statistically significant and measurable reduction in the biomarker level compared to the level of another biomarker or to a control level. The reduction is suitably a reduction of at least about 10%, or a reduction of at least about 20%, or a reduction of at least about 30%, or a reduction of at least about 40%, or a reduction of at least about 50%.

The term “saliva sample” as used herein includes any biological specimen that 25 may be extracted, untreated, treated, diluted or concentrated from a sample of saliva obtained from a subject. The term “saliva sample” includes saliva obtained from within the mouth, saliva obtained as spit, and saliva obtained from an oral rinse with a sampling fluid, such as sterile water.

The term “solid support” as used herein refers to a solid inert surface or body to which a molecular species, such as a nucleic acid and polypeptides can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

The terms “subject”, “individual” and “patient” are used interchangeably herein to refer to a normal healthy individual, or an individual in whom liver fibrosis or liver cirrhosis is absent, or any individual who may be at risk of liver fibrosis or liver cirrhosis, or suffering from liver fibrosis or liver cirrhosis, and/or has at least one clinical sign of liver fibrosis or liver cirrhosis.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, the term “treatment regimen” refers to prophylactic and/or therapeutic (i.e., after onset of a specified condition) treatments, unless the context specifically indicates otherwise. The term “treatment regimen” encompasses natural substances and pharmaceutical agents (i.e., “drugs”) as well as any other treatment regimen including but not limited to dietary treatments, physical therapy or exercise regimens, surgical interventions, radiotherapy, chemotherapy, immunotherapy and combinations thereof. Desirable effects of treatment include decreasing the rate of disease progression, ameliorating or palliating the disease state, and remission or improved prognosis. For example, an individual is successfully “treated” if one or more symptoms associated with liver fibrosis are mitigated or eliminated, including, but are not limited to, reducing viral infection, inhibiting fibrosis of the liver, inhibiting liver cirrhosis, decreasing symptoms resulting from liver fibrosis or cirrhosis of the liver, increasing the quality of life of those suffering from liver fibrosis or cirrhosis of the liver, decreasing the dose of other medications required to treat liver fibrosis or cirrhosis of the liver, and/or prolonging survival of individuals. The phrase “treatment with a therapy”, “treating with a therapy”, “treatment with an agent”, “treating with an agent” and the like refers to the administration of an effective amount of a therapy or agent, including a liver fibrosis or liver cirrhosis therapy or agent to a patient, or the concurrent administration of two or more therapies or agents in effective amounts to a patient.

It will be appreciated that the terms used herein and associated definitions are used for the purpose of explanation only and are not intended to be limiting.

### 2. Salivary Biomarkers for Determining Presence, Absence or Degree of Liver Fibrosis and Diagnosis of Liver Cirrhosis

Disclosed herein are methods, compositions, apparatuses, devices and kits for aiding in determining a presence, absence or degree of liver fibrosis and in assessing a likelihood that liver cirrhosis is present, absent or developing in a subject. These methods, compositions, apparatuses, devices and kits are useful for early detection of liver fibrosis and cirrhosis of the liver, thus allowing better treatment decisions for subjects with one or more clinical signs of liver fibrosis.

The present inventors have determined that certain biomarkers are commonly, specifically and differentially expressed in saliva samples obtained from healthy subjects, subjects with non-fibrotic liver disease, and subjects with liver fibrosis including subjects with cirrhosis of the liver. The results presented herein provide clear evidence that these specific biomarkers can be used to identify subjects with liver fibrosis and to diagnose a presence, absence, or risk of development of cirrhosis in affected individuals. The biomarkers that can be used in the practice of the methods, apparatuses and treatment methods disclosed herein include one or more of a glycosaminoglycan biomarker, hyaluronic acid (HA), and protein biomarkers: α-2-macroglobulin (A2MG), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS).

In various embodiments, the methods, compositions, apparatuses, devices and kits of the present disclosure are used to provide an indicator that aids in the diagnosis of liver fibrosis, including liver cirrhosis in a subject, suitably one with at least one clinical sign of liver fibrosis. In other embodiments, the disclosed methods, compositions, apparatuses, devices and kits are used as an aid to screen at risk patients for a presence, absence or severity of liver fibrosis, including liver cirrhosis.

In one aspect, methods are disclosed for determining an indicator used in assessing a likelihood of a subject having a presence, absence or degree of liver fibrosis, and in particular embodiments are used to determine an indicator for assessing a likelihood that liver cirrhosis is present, absent or developing in a subject. These methods generally comprise, consist or consist essentially of: (1) determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from A2MG, HA, TIMP1, CA1, KPNA3, and PGLS; and (2) determining the indicator using the biomarker value(s).

Biomarker values that are indicative of the levels of biomarkers in a saliva sample (also referred to herein as “salivary biomarkers”) may be obtained by any suitable means known in the art. A saliva sample can be saliva obtained from within the mouth, or obtained as spit. A saliva sample can also be a sample comprising saliva, as obtained by oral rinsing with a sampling rinse fluid, typically, e.g., sterile water, and then collecting the rinse, which then comprises saliva diluted with the rinse fluid.

Methods of obtaining saliva samples may include but are not limited to forcible ejection from the subject's mouth (e.g., spitting), aspiration, or removal by a swab or other collection tool. In some embodiments, the saliva may be separated into cellular and non-cellular fractions by suitable methods (e.g., centrifugation).

The level of the one or more biomarkers may be measured or assessed using any appropriate technique or means known to those of skill in the art. In particular embodiments, the level of a biomarker, such as A2MG, HA, TIMP1, CA1, KPNA3, and PGLS, is assessed using an antibody-based technique, non-limiting examples of which include immunoassays, such as the enzyme-linked immunosorbent assay (ELISA) and the radioimmunoassay (RIA). A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker. ELISAs for measuring the levels of A2MG, HA, TIMP1, CA1, KPNA3, and PGLS are available commercially from multiple sources and/or can be readily developed by those skilled in the art using known antibodies specific for A2MG, HA, TIMP1, CA1, KPNA3, and PGLS.

In specific embodiments, where the levels of two or more biomarkers are assessed, a multiplex assay, such as a multiplex immunoassay (e.g., multiplex ELISA), can be employed. Multiplex assays include arrays comprising spatially addressed antigen-binding molecules, commonly referred to as antibody arrays, which can facilitate extensive parallel analysis of multiple proteins or polysaccharides (e.g., glycosaminoglycans). Antibody arrays have been shown to have the required properties of specificity and acceptable background. Various methods for the preparation of antibody arrays have been reported (see, e.g., Lopez et al., J. Chromatogr. 2003; 787:19-27; Cahill, Trends Biotechnol. 2000; 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210).

Individual spatially distinct biomarker-capture agents (e.g., protein-capture agents, polysaccharide-capture agents) are typically attached to a support surface, which is generally planar or contoured. Common physical supports include glass slides, silicon, microwells, nitrocellulose or PVDF membranes, and magnetic and other microbeads.

Particles in suspension can also be used as the basis of multiplex assays and arrays, providing they are coded for identification; systems include color coding for microbeads (e.g., available from Luminex, Bio-Rad and Nanomics Biosystems) and semiconductor nanocrystals (e.g., QDots™, available from Quantum Dots), and barcoding for beads (UltraPlexT™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual biomarker-capture agents (e.g., protein-capture agents, polysaccharide-capture agents) are typically attached to an individual particle to provide the spatial definition or separation of the array. The particles may then be assayed separately, but in parallel, in a compartmentalized way, for example in the wells of a microtiter plate or in separate test tubes.

One illustrative example of a biomarker-capture array is Luminex™-based multiplex assay, which is a bead-based multiplexing assay, where beads are internally dyed with fluorescent dyes to produce a specific spectral address. Biomolecules (such as an antibody) can be conjugated to the surface of beads to capture biomarkers of interest. Flow cytometric or other suitable imaging technologies known to persons skilled in the art can then be used for characterization of the beads and detection and quantitation of the biomarkers.

In specific embodiments, multiplex assays use detectably distinct antibodies to distinctly label individual biomarkers.

Other methods for detecting and quantitating biomarkers include, but are not limited to, mass spectrometry (MS) methods, including Liquid Chromatography-Mass Spectrometry (LC-MS), Direct Analysis in Real Time Mass Spectrometry (DART MS), SELDI-TOF and MALDI-TOF, gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.).

In non-limiting embodiments of the assays of the present disclosure, compositions are prepared for use in the indicator-determining methods disclosed herein. These compositions may comprise a mixture of a saliva sample obtained from the subject, and for each of a plurality of biomarkers an antibody or antigen-binding fragment that binds specifically to the biomarker, wherein the plurality of biomarkers comprises, consists or consists essentially of A2MG, HA, TIMP1, CA1, KPNA3, and PGLS. Individual antibodies or antigen-binding fragments may have a label associated therewith. The antibodies may be directly labeled or are capable of being bound specifically by an ancillary affinity moiety (e.g., another antibody or antigen-binding fragment) that is labeled. In some embodiments, the composition comprises a plurality of antibodies or antigen-binding fragments, each of which specifically binds to a different biomarker and is associated with the same label or a different label, as compared to the biomarker specificity and label associated with other antibodies or antigen-binding fragments of the composition. In illustrative examples of this type, the labels associated with different antibodies or antigen-binding fragments are detectably distinct.

### 2.1 Analysis of Biomarker Data

Biomarker data may be analyzed by a variety of methods to identify salivary biomarkers and determine the statistical significance of differences in observed levels of biomarkers between test and reference salivary biomarker samples in order to evaluate whether a subject has a likelihood of having a presence, absence or degree of liver fibrosis or a likelihood of having a presence or absence of liver cirrhosis. For any particular biomarker, a distribution of biomarker levels or abundances for a first patient group (e.g., healthy subjects or subjects lacking liver fibrosis) and a second patient group (e.g., subjects with liver cirrhosis) will likely overlap. Under such conditions, a test does not absolutely distinguish the different groups with 100% accuracy, and the area of overlap indicates where the test cannot distinguish the first group and the second group. A threshold is selected, above which (or below which, depending on how biomarker changes with a specified condition) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)).

Alternatively, or in addition, thresholds may be established by obtaining an earlier biomarker result from the same patient, to which later results may be compared. In these embodiments, the individual in effect acts as their own “control group.” For biomarkers that increase inversely with liver fibrosis severity, an increase over time in the same patient can indicate a worsening of liver fibrosis or a failure of a treatment regimen or poor outcome, while a decrease over time can indicate remission of the condition or success of a treatment regimen or good outcome.

In some embodiments, a positive likelihood ratio, negative likelihood ratio, odds ratio, and/or AUC or receiver operating characteristic (ROC) values are used as a measure of a method's ability to predict risk or to diagnose a condition disclosed herein (e.g., liver fibrosis, liver cirrhosis, etc.). As used herein, the term “likelihood ratio” is the probability that a given test result would be observed in a subject with a condition of interest divided by the probability that that same result would be observed in a patient without the condition of interest. Thus, a positive likelihood ratio is the probability of a positive result observed in subjects with the specified condition (e.g., liver fibrosis, liver cirrhosis, etc.). A negative likelihood ratio is the probability of a negative result in subjects without the specified condition divided by the probability of a negative result in subjects with the specified condition. The term “odds ratio,” as used herein, refers to the ratio of the odds of an event occurring in one group (e.g., one of the disclosed conditions; e.g., healthy condition) to the odds of it occurring in another group (e.g., another of the disclosed conditions; e.g., liver fibrosis or liver cirrhosis), or to a data-based estimate of that ratio. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., one of the disclosed conditions and another of the disclosed conditions). ROC curves are useful for plotting the performance of a particular feature (e.g., any of the salivary biomarkers disclosed herein and/or any item clinical parameter or symptom information) in distinguishing or discriminating between two populations (e.g., one of the disclosed conditions and another of the disclosed conditions). Typically, the feature data across the entire population (e.g., subjects with one of the disclosed conditions and subjects with another of the disclosed conditions) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls. Although this definition refers to scenarios in which a feature is elevated in one patient group compared to another patient group, this definition also applies to scenarios in which a feature is lower in one patient group compared to the other patient group (in such a scenario, samples below the value for that feature would be counted). ROC curves can be generated for a single feature as well as for other single outputs, for example, a combination of two or more features (e.g., a combination of two or more biomarker values) can be mathematically combined (e.g., added, subtracted, multiplied, etc.) to produce a single value, and this single value can be plotted in a ROC curve. Additionally, any combination of multiple features (e.g., a combination of multiple biomarker values), in which the combination derives a single output value, can be plotted in a ROC curve. These combinations of features may comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one. An AUC ROC value may be thought of as equivalent to the Mann-Whitney U test, which tests for the median difference between scores obtained in the two groups considered if the groups are of continuous data, or to the Wilcoxon test of ranks.

In some embodiments, a panel of biomarkers (e.g., a panel comprising, consisting or consisting essentially of at least 1, 2, 3, 4, 5 05 biomarkers selected from A2MG, HA, TIMP1, CA1, KPNA3, and PGLS) is selected to discriminate between subjects with a first disclosed condition (e.g., healthy or non-fibrotic liver) and subjects with a second disclosed condition (e.g., liver cirrhosis), with at least about 50%, 55% 60%, 65%, 70%, 75%, 80%, 85%, 90%, 95% accuracy or having a C-statistic of at least about 0.50, 0.55, 0.60, 0.65, 0.70, 0.75, 0.80, 0.85, 0.90, 0.95.

In the case of a positive likelihood ratio, a value of 1 indicates that a positive result is equally likely among subjects in both the “first condition” and “second condition” groups; a value greater than 1 indicates that a positive result is more likely in the first condition group; and a value less than 1 indicates that a positive result is more likely in the second condition group. In this context, “first condition” group is meant to refer to a group having one characteristic (e.g., a first disclosed condition) and “second condition” group (e.g., a second disclosed condition) lacking the same characteristic. In the case of a negative likelihood ratio, a value of 1 indicates that a negative result is equally likely among subjects in both the “first condition” and “second condition” groups; a value greater than 1 indicates that a negative result is more likely in the “first condition” group; and a value less than 1 indicates that a negative result is more likely in the “second condition” group. In the case of an odds ratio, a value of 1 indicates that a positive result is equally likely among subjects in both the “first condition” and “second condition” groups; a value greater than 1 indicates that a positive result is more likely in the “first condition” group; and a value less than 1 indicates that a positive result is more likely in the “second condition” group. In the case of an AUC ROC value, this is computed by numerical integration of the ROC curve. The range of this value can be 0.5 to 1.0. A value of 0.5 indicates that a classifier (e.g., a biomarker signature) is no better than a 50% chance to classify unknowns correctly between two groups of interest (e.g., a first disclosed prognostic outcome and a second disclosed prognostic outcome disclosed herein), while 1.0 indicates the relatively best diagnostic accuracy. In certain embodiments, biomarker panels are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, biomarker panels are selected to exhibit an odds ratio 35 of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less.

In certain embodiments, biomarker panels are selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds may be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” may be considered “thresholds.” A risk (of a particular diagnosis or prognosis for example) can be assigned based on which “bin” a test subject falls into.

In other embodiments, particular thresholds for the biomarker(s) measured are 5 not relied upon to determine if the biomarker level(s) obtained from a subject are correlated to a particular prognosis. For example, a temporal change in the biomarker(s) can be used to rule in or out one or more particular diagnoses. Alternatively, biomarker(s) may be correlated to a condition by a presence or absence of one or more biomarkers in a particular assay format. In the case of biomarker panels, the detection methods disclosed herein may utilize an evaluation of the entire population or subset of biomarkers disclosed herein to provide a single result value (e.g., a “panel response” value expressed either as a numeric score or as a percentage risk).

In certain embodiments, a panel of biomarkers (e.g., a panel comprising, consisting or consisting essentially of at least 1, 2, 3, 4, 5 05 biomarkers selected from A2MG, HA, TIMP1, CA1, KPNA3, and PGLS) is selected to assist in distinguishing a pair of groups (i.e., assist in assessing whether a subject has an increased likelihood of being in one group or the other group of the pair) selected from “healthy” on “non-fibrotic liver” and “liver cirrhosis”, or “low risk” and “high risk” with at least about 70%, 80%, 85%, 90% or 95% sensitivity, suitably in combination with at least about 70% 80%, 85%, 90% or 95% specificity. In some embodiments, both the sensitivity and specificity are at least about 75%, 80%, 85%, 90% or 95%.

The phrases “assessing the likelihood” and “determining the likelihood,” as used herein, refer to methods by which the skilled artisan can predict a presence, absence or risk of development of a condition (e.g., a condition selected from “healthy”, “non-fibrotic liver” and “fibrotic liver” (e.g., “intermediate fibrosis” or “liver cirrhosis”). The skilled artisan will understand that this phrase includes within its scope an increased probability that a condition is present, absent or developing in a patient; that is, that a condition is more likely to be present, absent or developing in a subject. For example, the probability that an individual identified as having a specified condition actually has the condition may be expressed as a “positive predictive value” or “PPV.” Positive predictive value can be calculated as the number of true positives divided by the sum of the true positives and false positives. PPV is determined by the characteristics of the predictive methods disclosed herein as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition actually does not have that condition may be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 7/5%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined as having a significant likelihood of having or not having a specified condition (e.g., “healthy condition”/“non-fibrotic liver condition”, “liver fibrosis” or “liver cirrhosis”). By “significant likelihood” is meant that the subject has a reasonable probability (0.6, 0.7, 0.8, 0.9 or more) of having, or not having, a specified condition or prognosis.

The biomarker analysis disclosed herein permits the generation of high-density data sets that can be evaluated using informatics approaches. High data density informatics analytical methods are known and software is available to those in the art, e.g., cluster analysis (Pirouette, Informetrix), class prediction (SIMCA-P, Umetrics), principal components analysis of a computationally modeled dataset (SIMCA-P, Umetrics), 2D cluster analysis (GeneLinker Platinum, Improved Outcomes Software), and metabolic pathway analysis (biotech.icmb.utexas.edu). The choice of software packages offers specific tools for questions of interest (Kennedy et al., Solving Data Mining Problems Through Pattern Recognition. Indianapolis: Prentice Hall PTR, 1997; Golub et al., (2999) Science 286:531-7; Eriksson et al., Multi and Megavariate Analysis Principles and Applications: Umetrics, Umea, 2001). In general, any suitable mathematic analyses can be used to evaluate a panel of biomarkers disclosed herein with respect to a disclosed condition (e.g., a “healthy condition”/“non-fibrotic liver condition”, “liver fibrosis” or “liver cirrhosis”). For example, methods such as multivariate analysis of variance, multivariate regression, and/or multiple regression can be used to determine relationships between dependent variables (e.g., clinical measures) and independent variables (e.g., levels of biomarkers). Clustering, including both hierarchical and non-hierarchical methods, as well as non-metric Dimensional Scaling can be used to determine associations or relationships among variables and among changes in those variables.

In addition, principal component analysis is a common way of reducing the dimension of studies, and can be used to interpret the variance-covariance structure of a data set. Principal components may be used in such applications as multiple regression and cluster analysis. Factor analysis is used to describe the covariance by constructing “hidden” variables from the observed variables. Factor analysis may be considered an extension of principal component analysis, where principal component analysis is used as parameter estimation along with the maximum likelihood method. Furthermore, simple hypothesis such as equality of two vectors of means can be tested using Hotelling's T squared statistic.

In some embodiments, the data sets corresponding to biomarker panels disclosed herein are used to create a predictive rule or model based on the application of a statistical and machine learning algorithm. Such an algorithm uses relationships between a biomarker panel and a disclosed condition (e.g., a “healthy condition”/“non-fibrotic liver condition”, 35 “liver fibrosis” or “liver cirrhosis”), observed in control subjects or typically cohorts of control subjects (sometimes referred to as training data), which provides combined control or reference biomarker panels for comparison with biomarker panels of a subject. The data are used to infer relationships that are then used to predict the status of a subject, including a presence, absence or risk of development of one of the conditions referred to herein.

Practitioners skilled in the art of data analysis recognize that many different forms of inferring relationships in the training data may be used without materially changing the detection methods disclosed herein. The data presented in the Tables, Examples and Figures herein have been used to generate illustrative minimal combinations of biomarkers (models) that differentiate between the disclosed conditions (i.e., a “healthy condition”/“non-fibrotic liver condition” (i.e., “non-fibrotic condition”) or “intermediate fibrosis of the liver”/“liver cirrhosis” (i.e., “fibrotic liver condition”)) using feature selection based on AUC maximization in combination with analytical model classification, including for example classification using one or more of: an additive model; a linear model; a support vector machine; a neural network model; a random forest model; a regression model; a genetic algorithm; an annealing algorithm; a weighted sum; a nearest neighbor model; and a probabilistic model. The biomarkers disclosed herein provide illustrative lists of biomarkers ranked according to their p value. Illustrative models comprising a plurality of biomarkers disclosed herein were able to develop a classifier or generative algorithm for discriminating between “non-fibrotic condition” and “fibrotic liver condition” as defined above with significantly improved positive predictive values compared to conventional methodologies. This algorithm (also referred to herein as SALF score) can be advantageously applied to determine presence or probability of one of the conditions disclosed herein is present in a patient, and thus diagnose the patient as having or as likely to have the condition.

In some embodiments, evaluation of biomarkers includes determining the levels of individual biomarkers, which correlate with a condition, as defined above. In certain embodiments, the techniques used for detection of biomarkers may include internal or external standards to permit quantitative or semi-quantitative determination of those biomarkers, to thereby enable a valid comparison of the level of the biomarkers in a saliva sample with the corresponding biomarkers in a reference sample or samples. Such standards can be determined by the skilled practitioner using standard protocols. In specific examples, absolute values for the level or functional activity of individual expression products are determined.

In semi-quantitative methods, a threshold or cut-off value is suitably determined, and is optionally a predetermined value. In particular embodiments, the threshold value is predetermined in the sense that it is fixed, for example, based on previous experience with the assay and/or a population of affected and/or unaffected subjects. Alternatively, the predetermined value can also indicate that the method of arriving at the threshold is predetermined or fixed even if the particular value varies among assays or may even be determined for every assay run.

In some embodiments, the level of a biomarker is normalized. There is no intended limitation on the methodology used to normalize the values of the measured biomarkers provided that the same methodology is used for testing a human subject sample as was used to generate a risk categorization table or threshold value. Many methods for data normalization exist and are familiar to those skilled in the art. These include methods such as background subtraction, scaling, MoM analysis, linear transformation, least squares fitting, etc. The goal of normalization is to equate the varying measurement scales for the separate biomarkers such that the resulting values may be combined according to a weighting scale as determined and designed by the user or by the machine learning system and are not influenced by the absolute or relative values of the biomarker found within nature.

Composite scores may be calculated using standard statistical analysis well known to one of skill in the art wherein the measurements of each biomarker in the panel are combined, optionally with clinical parameters, to provide a probability value. For example, generalized or multivariate logistic regression analysis may be used to derive a mathematical function with a set of variables corresponding to each biomarker and optional clinical parameter, which provides a weighting factor for each variable. The weighting factors are derived to optimize the agency of the function to predict the dependent variable, which is the dichotomy of a first condition (e.g., “non-fibrotic condition”) as compared to a second condition (e.g., “fibrotic liver condition”) disclosed herein. The weighting factors are specific to the particular variable combination (e.g., biomarker panel analyzed). The function can then be applied to the original samples to predict a probability of a disclosed condition. In this way, a retrospective data set may be used to provide weighting factors for a particular panel of salivary biomarkers, optionally in combination with clinical parameters, which is then used to calculate the probability of a disclosed condition in a patient where the outcome of the condition is unknown or indeterminate prior to screening using the present methods.

Composite scores may be calculated for example using the statistical methodology disclosed in US Publ. No. 2008/013314 for handling and interpreting data from a multiplex assay. In this methodology, the amount of any one biomarker is compared to a predetermined cut-off distinguishing positive from negative for that biomarker as determined from a control population study of patients with a specified condition (e.g., intermediate liver fibrosis/liver cirrhosis) and suitably matched controls (e.g., healthy patients or patients without a fibrotic liver condition) to yield a score for each biomarker based on that comparison; and then combining the scores for each biomarker to obtain a composite score for the biomarker(s) in the sample.

A predetermined cut-off can be based on ROC curves and the score for each biomarker can be calculated based on the specificity of the biomarker. Then, the total score can be compared to a predetermined total score to transform that total score to a qualitative determination of the likelihood or risk of having a condition as disclosed herein.

In certain embodiments, the biomarkers disclosed herein are measured and those resulting values normalized and then summed to obtain a composite score. In certain aspects, normalizing the measured biomarker values comprises determining the multiple of median (MoM) score. In other aspects, the present method further comprises weighting the normalized values before summing to obtain a composite score. In illustrative examples of this type, the median value of each biomarker is used to normalize all measurements of that specific biomarker, for example, as provided in Kutteh et al. (Obstet. Gynecol. 1994; 84:811-815) and Palomaki et al. (Clin. Chem. Lab. Med 2001; 39:1137-1145). Thus, any measured biomarker level is divided by the median value of a disclosed condition group (e.g., “healthy condition”/“non-fibrotic liver condition” or “liver cirrhosis”), resulting in a MoM value. The MoM values can be combined (namely, summed or added) for each biomarker in the panel resulting in a panel MoM value or aggregate MoM score for each sample.

If desired, a machine learning system may be utilized to determine weighting of the normalized values as well as how to aggregate the values (e.g., determine which biomarkers are most predictive, and assign a greater weight to these biomarkers).

In specific embodiments, a composite score for determining an indicator used in assessing a likelihood of having a disclosed condition is determined by a statistical model based on analyzing biomarker (e.g., protein biomarker and/or polysaccharide biomarker) significance by applying a linear mixed-effects model using MSstats, as described previously (Zhang et al., Theranostics. 2017; 7(18):4350-8). This analysis consisted of quantitative measurements for a targeted biomarker based on peptides, charge states, transitions, samples, and conditions. The method identifies biomarker alterations in abundance between conditions more systematically than random chance (Zhang et al., 2017; supra). The biomarker abundance levels between patients with unfavorable and favorable outcomes were compared using the Mann-Whitney test (GraphPad Prism). A p value <0.05 was defined as statistically significant.

In specific embodiments, a composite score for determining an indicator used in assessing a likelihood of having a presence or absence of liver fibrosis (e.g., intermediate degree of liver fibrosis) or liver cirrhosis is determined using the following algorithm:

$$y = EXP [ - 15.8454816 + ( HA \times 0.7944629 ) + ( TIMP 1 \times 1.3469354 ) + ( A 2 MG \times 0.1541859 ) ]$$

$$wherein :$$

$$SALF = \frac{y}{1 + y}$$

In other embodiments, a composite score for determining an indicator used in assessing a likelihood of having a presence or absence of liver fibrosis (i.e., intermediate degree of liver fibrosis) or liver cirrhosis is determined using the following algorithm:

$$y = EXP [ - 11.3995405 + ( HA \times 1.1074823 ) - ( TIMP 1 \times 1.0547269 ) + ( A 2 MG \times 0.1504837 )$$

$$wherein :$$

$$SALF = \frac{y}{1 + y}$$

In still other embodiments, a composite score for determining an indicator used in assessing a likelihood of having a presence or absence of liver fibrosis (i.e., intermediate degree of liver fibrosis) or liver cirrhosis is determined using the following algorithm:

$$y = EXP [ - 1.78007 + ( HA \times 0. 1132 ) + ( TIMP 1 \times 0.0431 ) + ( A 2 MG \times 0.05163 ) ]$$

$$wherein :$$

$$SALF = \frac{y}{1 + y}$$

In other embodiments, a composite score for determining an indicator used in assessing a likelihood of having a presence or absence of liver fibrosis (i.e., intermediate degree of liver fibrosis) or liver cirrhosis is determined using the following algorithm:

$$y = EXP [ - 2.139255264 + ( - 649329.5 \; 654 \times KPNA 3 ) + ( 180500.0745 \times PGLS ) + ( A 51528.8054 \times CA 1 ) ]$$

$$wherein :$$

$$SALF = \frac{y}{1 + y}$$

In still other embodiments, a composite score for determining an indicator used in assessing a likelihood of having a presence or absence of liver fibrosis (i.e., intermediate degree of liver fibrosis) or liver cirrhosis is determined using the following algorithm:

$$y = EXP [ \text{} - 1.944362 + ( HA \times 0.1132 ) + ( TIMP 1 \times 0.0431 ) + ( A 2 MG \times 0.05163 ) + ( CA 1 \times 15040.0736 ) + ( PGLS \times 40235.0491 ) + ( KPNA 3 \times - 52432.74609 ) ]$$

$$wherein :$$

$$SALF = \frac{y}{1 + y}$$

In non-limiting examples, the cut-off score for y is 0.514, wherein a score of greater than 0.514 is indicative of a likelihood of a presence of liver fibrosis (e.g., intermediate degree of liver fibrosis) or liver cirrhosis, and wherein a score of less than 0.514 is indicative of a likelihood of an absence of liver fibrosis (e.g., intermediate degree of liver fibrosis) or liver cirrhosis.

In some embodiments, composite scores include one or more clinical parameters or signs of the patient. Representative clinical parameters or signs include age, ethnicity, gender, Hepatitis virus (e.g., Hepatitis B or Hepatitis C) antigen, Hepatitis virus (e.g., Hepatitis B or Hepatitis C) nucleic acid, alanine aminotransferase (ALT}level, alkaline phosphatase(ALP) level, platelet count, standard deviation of red blood cell distribution width (RDW-SD), albumin level, bilirubin level, γ-glutamyl transpeptidase (GGT) level and α-fetoprotein (AFP) level.

In certain embodiments, the detection methods utilize a risk categorization table to generate a risk score for a patient based on a composite score by comparing the composite score with a reference set derived from a cohort of patients with one of the conditions disclosed herein. The detection methods may further comprise quantifying the increased risk for a presence or risk of development of a disclosed condition in the patient as a risk score, wherein the composite score (combined obtained biomarker value and optionally obtained clinical parameter values) is matched to a risk category of a grouping of stratified patient populations wherein each risk category comprises a multiplier (or percentage) indicating an increased likelihood of having the condition correlated to a range of composite scores. This quantification is based on the pre-determined grouping of a stratified cohort of subjects. In some embodiments, the grouping of a stratified population of subjects, or stratification of a prognosis cohort, is in the form of a risk categorization table. The selection of the disease cohort, the cohort of subjects that share disclosed condition risk factors, are well understood by those skilled in the art of cancer research. However, the skilled person would also recognize that the resulting stratification, may be more multidimensional and take into account further environmental, occupational, genetic, or biological factors (e.g., epidemiological factors).

After quantifying the increased risk for presence of a condition (e.g., “non-fibrotic condition” and “fibrotic liver condition”) in the form of a risk score, this score may be provided in a form amenable to understanding by a physician. In certain embodiments, the risk score is provided in a report. In certain aspects, the report may comprise one or more of the following: patient information, a risk categorization table, a risk score relative to a cohort population, one or more biomarker test scores, a biomarker composite score, a master composite score, identification of the risk category for the patient, an explanation of the risk categorization table, and the resulting test score, a list of biomarkers tested, a description of the disease cohort, environmental and/or occupational factors, cohort size, biomarker velocity, genetic mutations, family history, margin of error, and so on.

In some embodiments, a subject whose risk score is indicative of a likelihood of a presence of a fibrotic liver condition (e.g., intermediate liver fibrosis or liver cirrhosis) is further assessed using an ancillary liver fibrosis detection technique to confirm that the subject has liver fibrosis (e.g., intermediate liver fibrosis or liver cirrhosis). Representative examples of such detection techniques include liver biopsy, liquid biopsy, ultrasound imaging, elastography, and serum biomarkers, such as the OWLiver Test from Owl Metabolomics, 13C-methacetin breath test (MBT) from Exalenz Bioscience, Plasma Pro-C3 from Nordic Bioscience, Fibroscan from Echosens for transient elastography (TE) using ultrasound, Magnetic Resonance Elastography (MRE) by Resoundant, Inc., and LiverMultiScan from Perspectum Diagnostics.

### 3. Kits

All the essential reagents required for detecting and quantifying the biomarkers disclosed herein may be assembled together in a kit. In some embodiments, the kit comprises a reagent that permits quantification of each biomarker of a biomarker panel disclosed herein. In the context of the present disclosure, “kit” is understood to mean a product containing the different reagents necessary for carrying out the methods of the disclosure packed so as to allow their transport and storage. Additionally, the kits of the present disclosure can contain instructions for the simultaneous, sequential or separate use of the different components contained in the kit. The instructions can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain internet addresses that provide the instructions. The kits may contain software for interpreting assay data to determine the likelihood of a subject having a presence, absence or degree of liver fibrosis, or to determine a likelihood that liver cirrhosis is present, absent or developing in a subject. In some embodiments, the kits may provide a means to access a machine learning system provided, for example, as a software as a service (SaaS) deployment.

Reagents that allow quantification of biomarkers include compounds or materials, or sets of compounds or materials, which allow quantification of the biomarkers. In specific embodiments, the compounds, materials or sets of compounds or materials permit determining the level or abundance of biomarkers (e.g., the salivary biomarkers disclosed herein) include without limitation the isolation or preparation of a protein and/or polysaccharide sample from a saliva sample, the determination of the level of a corresponding biomarker, etc., antibodies for specifically binding to disclosed biomarkers, etc.

Kit reagents can be in liquid form or can be lyophilized. Suitable containers for the reagents include, for example, bottles, vials, syringes, and test tubes. Containers can be formed from a variety of materials, including glass or plastic. The kit can also comprise a package insert containing written instructions for methods of diagnosing a condition disclosed herein or prognosis patient survival.

The kits may also optionally include appropriate reagents for detection of labels, positive and negative controls, washing solutions, blotting membranes, microtiter plates, dilution buffers and the like. The kit can also feature various devices (e.g., one or more) and reagents (e.g., one or more) for performing one of the assays described herein; and/or printed instructions for using the kit to quantify at least one biomarker disclosed herein and/or carry out an indicator-determining method, as broadly described above and elsewhere herein.

The reagents described herein, which may be optionally associated with detectable labels, can be presented in the format of a microfluidics card, a reaction vessel, a microarray or a kit adapted for use with the assays described in the examples.

### 4. Device Embodiments

Also contemplated herein are embodiments in which the indicator-determining methods of the invention are implemented using one or more processing devices. In representative embodiments of this type, a disclosed method that is implemented by the processing device(s) determines an indicator used in assessing a likelihood of a subject having a presence, absence or degree of liver fibrosis, or having a presence or absence of a disclosed condition (e.g., “healthy condition”/“non-fibrotic liver condition” or “liver cirrhosis”) wherein the method comprises: (1) determining a biomarker value for determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from A2MG, HA, TIMP1, CA1, KPNA3, and PGLS; (2) determining the indicator using the biomarker value(s); (3) retrieving previously determined indicator references from a database, the indicator references being determined based on indicators determined from a reference population consisting of individuals diagnosed with a presence, absence or degree of liver fibrosis or diagnosed with presence of the disclosed condition; (4) comparing the indicator to the indicator references to thereby determine a probability indicative of the subject having or not having a presence, absence or degree of liver fibrosis, or having or not having the disclosed condition; and (5) generating a representation of the probability, the representation being displayed to a user to allow the user to assess the likelihood of the subject having the condition or survival prognosis.

In specific embodiments, an apparatus is provided for determining the likelihood of a subject having a presence, absence or degree of liver fibrosis, or having a presence or absence of a disclosed condition (e.g., “healthy condition”/“non-fibrotic liver condition” or “liver cirrhosis”). The apparatus typically includes at least one electronic processing device that:
determines a biomarker value for for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from A2MG, HA, TIMP1, CA1, KPNA3, and PGLS; and
determines the indicator using the biomarker value(s).

The apparatus may further include any one or more of:
(A) a sampling device that obtains a saliva sample taken from a subject, the saliva sample including a plurality of biomarkers disclosed herein;
(B) a measuring device that quantifies for each of the salivary biomarkers a corresponding biomarker value;
(C) at least one processing device that:
   (i) receives the biomarker values from the measuring device;
   (ii) determines an indicator that is indicative of a presence, absence or degree of liver fibrosis, or a presence, absence or risk of development of liver cirrhosis, or of a healthy condition or non-fibrotic liver condition using the biomarker values optionally in combination with one or more clinical parameters or signs of the subject;
   (iii) compares the indicator to at least one indicator reference;
   (iv) determines a likelihood of the subject having or not having a presence or degree of liver fibrosis, or having or not having a presence or risk of development of liver cirrhosis using the results of the comparison; and
   (v) generates a representation of the indicator and the likelihood for display to a user.

In some embodiments, the apparatus comprises a processor configured to execute computer readable media instructions (e.g., a computer program or software application, e.g., a machine learning system, to receive the biomarker values from the evaluation of biomarkers in a sample and, in combination with other risk factors (e.g., medical history of the patient, publically available sources of information pertaining to a risk of liver fibrosis or liver cirrhosis) may determine a master composite score and compare it to a grouping of stratified cohort population comprising multiple risk categories (e.g., a risk categorization table) and provide a risk score. Methods and techniques for determining a master composite score and a risk score are known in the art.

The apparatus can take any of a variety of forms, for example, a handheld device, a tablet, or any other type of computer or electronic device. The apparatus may also comprise a processor configured to execute instructions (e.g., a computer software product, an application for a handheld device, a handheld device configured to perform the method, a world-wide-web (WWW) page or other cloud or network accessible location, or any computing device. In other embodiments, the apparatus may include a handheld device, a tablet, or any other type of computer or electronic device for accessing a machine learning system provided as a software as a service (SaaS) deployment. Accordingly, the correlation may be displayed as a graphical representation, which, in some embodiments, is stored in a database or memory, such as a random access memory, read-only memory, disk, virtual memory, etc. Other suitable representations, or exemplifications known in the art may also be used.

The apparatus may further comprise a storage means for storing the correlation, an input means, and a display means for displaying the status of the subject in terms of a presence, absence or degree of liver fibrosis, or a presence, absence of the disclosed condition. The storage means can be, for example, random access memory, read-only memory, a cache, a buffer, a disk, virtual memory, or a database. The input means can be, for example, a keypad, a keyboard, stored data, a touch screen, a voice-activated system, a downloadable program, downloadable data, a digital interface, a hand-held device, or an infrared signal device. The display means can be, for example, a computer monitor, a cathode ray tube (CRT), a digital screen, a light-emitting diode (LED), a liquid crystal display (LCD), an X-ray, a compressed digitized image, a video image, or a hand-held device. The apparatus can further comprise or communicate with a database, wherein the database stores the correlation of factors and is accessible to the user.

In certain embodiments, the apparatus is a computing device, for example, in the form of a computer or hand-held device that includes a processing unit, memory, and storage. The computing device can include, or have access to a computing environment that comprises a variety of computer-readable media, such as volatile memory and non-volatile memory, removable storage and/or non-removable storage. Computer storage includes, for example, RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output, and/or a communication connection. The input can be one or several devices, such as a keyboard, mouse, touch screen, or stylus. The output can also be one or several devices, such as a video display, a printer, an audio output device, a touch stimulation output device, or a screen reading output device. If desired, the computing device can be configured to operate in a networked environment using a communication connection to connect to one or more remote computers. The communication connection can be, for example, a Local Area Network (LAN), a Wide Area Network (WAN) or other networks and can operate over the cloud, a wired network, wireless radio frequency network, and/or an infrared network.

### 5. Treatment Management Embodiments

The indicator-determining methods, apparatuses, composition and kits of the present disclosure are useful for managing treatment decisions for liver fibrosis or liver cirrhosis, including managing the development or progression liver fibrosis, including liver cirrhosis, in a subject. A subject positively identified, optionally with an ancillary liver fibrosis detection method, as having liver fibrosis, including liver cirrhosis, may be managed by a treatment regimen, including treatment regimens that are suited to treating the severity of fibrosis indicated by the indicator-determining methods.

For example, patients with low to moderate (intermediate) levels of fibrosis may be directed to modify their behavior or routine to treat an underlying cause of liver fibrosis, illustrative examples of which include hepatitis viral infection, a hepatotoxicity, a non-alcoholic fatty liver disease (NAFLD), an autoimmune disease, a metabolic liver disease and a disease with secondary involvement of the liver. Depending on the underlying cause, non-limiting behavior or routine modifications include stopping or limiting alcohol use and using supportive therapies to help with this, treating chronic viral hepatitis infections with antiviral medications, treating NAFLD and NASH by balancing the diet, losing at least 7% body weight over 1 year, and controlling blood levels of fat, cholesterol, and sugar; taking medications that remove heavy metals, such as iron and copper, from the body, dissolving or removing bile duct obstructions, stopping the use of medications linked with fibrosis, and taking medications that reduce the activity of the immune system.

Alternatively, patients with higher levels of fibrosis, including with cirrhosis of the liver, may be administered at least one therapeutic agent or started on a complication screening program for applying early prophylactic or curative treatment. Non-limiting examples of therapeutic agents include, but are not limited to, bezafibrate, S-adenosyl-L-methionine, S-nitrosol-N-acetylcystein, silymarin, phosphatidylcholine, N-acetylcysteine, resveratrol, vitamin E, pentoxyphilline (or pentoxyfilline) alone or in combination with tocopherol, pioglitazone alone or in combination with vitamin E, lovaza (fish oil), PPC alone or in combination with an antiviral therapy (e.g., IFN), INT747, peginterferon 2b (pegylated IFNa-2b), a combination of infliximab, and ribavirin, stem cell transplantation (in particular MSC transplantation), candesartan, losartan, telmisartan, irbesartan, ambrisentan, FG-3019, Phyllanthus urinaria, Fuzheng Huayu, warfarin, insulin, colchicine, corticosteroids, naltrexone, RF260330, sorafenib, imatinib mesylate, nilotinib, pirfenidone, halofuginone, polaorezin, gliotoxin, sulfasalazine, rimonabant, simtuzumab, GR-MD-02, boceprevir, telaprevir, simeprevir, sofosbuvir, daclatasvir, elbasvir, grazoprevir, velpatasvir, lamivudine, adefovir dipivoxil, entecavir, telbivudine, tenofovir, clevudine, ANA380, zadaxin, CMX 157, ARB-1467, ARB-1740, ALN-HBV, BB-HB-331, Lunar-HBV, ARO-HBV, Myrcludex B, GLS4, NVR 3-778, AIC 649, JNJ56136379, ABI-H0731, AB-423, REP 2139, REP 2165, GSK3228836, GSK33389404, RNaseH Inhibitor, GS 4774, INO-1800, HB-110, TG1050, HepTcell, TomegaVax HBV, RG7795, SB9200, EYP001, CPI 431-32, topiramate, disulfiram, naltrexone, acamprosate, 35 baclofen, methadone, buprenorphine, orlistat, metformin, atorvastatin, ezetimine, ARBs, EPL, EPA-E, multistrain biotic (L. rhamnosus, L. bulgaricus), obeticholic acid, elafibranor (GFT505), DUR-928, GR-MD, 02, aramchol, RG-125, cenicriviroc CVC, rosiglitazone, MSDC-0602K, GS-9674, LJN452, LMB763, EDP-305, elafibranor, saroglitazar, IVA337, NGM282, PF-05231023, BMS-986036, aramchol, volixibat, GS-0976, liraglutide, semaglutide exenatide, taspoglutide, taurine, polyenephosphatidylcholine, MGL-3196, vitamin C, GS-4997, sitagliptin, alogliptin, vildagliptin, saxagliptin, linagliptin, PXS-4728A, VLX-103, hyperimmune bovine clostrum, nalmefene, emricasan, milk thistle; and probiotics and combinations thereof. In specific embodiments, the at least one therapeutic agent is an antifibrotic agent selected from the group consisting of simtuzumab, GR-MD-02, stem cell transplantation (in particular MSC transplantation), Phyllanthus urinaria, Fuzheng Huayu, S-adenosyl-L-methionine, S-nitrosol-N-acetylcystein, silyrnarin, phosphatidylcholine, N-acetylcysteine, resveratrol, vitamin E, losartan, telmisartan, naltrexone, RF260330, sorafenib, imatinib mesylate, nilotinib, INT747, FG-3019, oltipraz, pirfenidone, halofuginone, polaorezin, gliotoxin, sulfasalazine, rimonabant and combinations thereof.

In some embodiments, the underlying cause responsible for liver fibrosis is a viral infection and the at least one therapeutic agent is selected from the group consisting of interferon, peginterferon 2b (pegylated IFNa-2b), infliximab, ribavirin, boceprevir, telaprevir, simeprevir, sofosbuvir, daclatasvir, elbasvir, grazoprevir, velpatasvir, lamivudine, adefovir dipivoxil, entecavir, telbivudine, tenofovir, clevudine, ANA380, zadaxin, CMX 157, ARB-1467, ARB-1740, ALN-HBV, BB-HB-331, Lunar-HBV, ARO-HBV, Myrcludex B, GLS4, NVR 3-778, AIC 649, JNJ56136379, ABI-H0731, AB-423, REP 2139, REP 2165, GSK3228836, GSK33389404, RNaseH Inhibitor, GS 4774, INO-1800, HB-110, TG1050, HepTcell, TomegaVax HBV, RG7795, SB9200, EYP001, CPI 431-32 and combinations thereof.

In other embodiments, the underlying cause responsible for liver fibrosis is 15 excessive alcohol consumption and the at least one therapeutic agent is selected from the group consisting of topiramate, disulfiram, naltrexone, acamprosate and baclofen.

In still other embodiments, the underlying cause responsible for liver fibrosis is a non-alcoholic fatty liver disease (NAFLD) and the at least one therapeutic agent is selected from the group consisting of telmisartan, orlistat, metformin, pioglitazone, atorvastatin, ezetimine, vitamin E, sylimarine, pentoxyfylline, ARBs, EPL, EPA-E, multistrain biotic (L. rhamnosus, L. bulgaricus), simtuzumab, obeticholic acid, elafibranor (GFT505), DUR-928, GR-MD, 02, aramchol, RG-125, cenicriviroc CVC and combinations thereof.

In further embodiments, the underlying cause responsible for liver fibrosis is a nonalcoholic steatohepatitis (NASH), preferably fibrotic NASH, and the at least one therapeutic agent is selected from the group consisting of insulin sensitizers (such as rosiglitazone, pioglitazone and MSDC-0602K); farnesoid X receptor (FXR) agonists (such as obeticholic acid (also referred to as OCA), GS-9674, LJN452, LMB763 and EDP-305); Peroxisome Proliferator-Activated Receptor a/6 (PPAR a/6) agonists (such as elafibranor, saroglitazar and IVA337); fibroblast growth factor 19 (FGF19) analogs (such as NGM282); fibroblast growth factor 21 (FGF21) analogs (such as PF-05231023); recombinant FGF21 (such as BMS-986036); stearoyl-coenzyme A desaturase 1 (SCD1) inhibitors (such as aramchol); apical sodium-dependent bile acid transporter (ASBT) inhibitors (such as volixibat); acetyl-coA carboxylase (ACC) inhibitors (such as GS-0976); glucagon-like peptide-1 (GLP-1) analogs (such as liraglutide, semaglutide exenatide and taspoglutide); ursodeoxycholic acid and norursodeoxycholic acid (NorUDCA); taurine; polyenephosphatidylcholine; thyroid hormone receptor (THR) R-agonists (such as MGL-3196); antioxidant agents (such as vitamin E and vitamin C); apoptosis signal-regulating kinase 1 (ASK1) inhibitors (such as GS-4997); DPP-4 inhibitors (such as sitagliptin, alogliptin, vildagliptin, saxagliptin, and linagliptin); vascular adhesion protein-1 (VAP-1) inhibitors (such as PXS-4728A); phosphodiesterase-4 (PDE-4) inhibitors; angiotensin II-1 type receptor antagonists (such as losartan and telmisartan); anti-inflammatory compounds (such as cenicriviroc, VLX-103 (oral pentamidine) and hyperimmune bovine clostrum); Toll-like receptor 4 antagonists (such as nalmefene); caspase inhibitors (such as emricasan); pentoxifylline; S-adenosylmethionine; milk thistle; and probiotics.

Fibrosis severity of patients may be monitored at regular intervals to determine whether a treatment regimen is effective in treating the fibrosis. In some embodiments, fibrosis severity is assessed every 3 months, every 6 months, every 9 months, every 12 months, every 15 months, every 18 months, every 24 months, or every 36 months.

### 6. Representative Embodiments of the Disclosure

1. A method for determining an indicator used in assessing a likelihood of a subject having a presence, absence or degree or severity of liver fibrosis, the method comprising, consisting or consisting essentially of:
   (1) determining a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of α-2-macroglobulin (A2MG), hyaluronic acid (HA), and tissue inhibitor matrix metalloproteinase 1 (TIMP1); and
   (2) determining the indicator using the biomarker values.
2. A method for determining an indicator used in assessing a likelihood that liver cirrhosis is present, absent or developing in a subject, the method comprising, consisting or consisting essentially of:
   (1) determining a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of α-2-macroglobulin (A2MG), hyaluronic acid (HA), and tissue inhibitor matrix metalloproteinase 1 (TIMP1); and
   (2) determining the indicator using the biomarker values.
3. A method for determining an indicator used in assessing a likelihood of a subject having a presence, absence or degree or severity of liver fibrosis, the method comprising, consisting or consisting essentially of:
   (1) determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS); and
   (2) determining the indicator using the biomarker value(s)s.
4. A method for determining an indicator used in assessing a likelihood that liver cirrhosis is present, absent or developing in a subject, the method comprising, consisting or consisting essentially of:
   (1) determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from A2MG, HA, TIMP1, CA1, KPNA3, and PGLS; and
   (2) determining the indicator using the biomarker value(s).
5. The method of embodiment 3 or embodiment 4, wherein the at least one biomarker is represented by a biomarker signature selected from: [A2MG]; [HA]; [TIMP1]; [CA1]; [KPNA3]; [PGLS]; [A2MG:HA]; [A2MG:TIMP1]; [A2MG:CA1]; [A2MG:KPNA3]; [A2MG:PGLS]; [HA:TIMP1]; [HA:CA1]; [HA:KPNA3]; [HA:PGLS]; [TIMPi:CA1]; [TIMPi:KPNA3]; [TIMPi:PGLS]; [CA1:KPNA3]; [CA1:PGLS]; [KPNA3:PGLS]; [A2MG:HA:TIMP1]; [A2MG:HA:CA1]; [A2MG:HA:KPNA3]; [A2MG:HA:PGLS]; [A2MG:TIMP1:CA1]; [A2MG:TIMP1:KPNA3]; [A2MG:TIMP1:PGLS]; [A2MG:CA1:KPNA3]; [A2MG:CA1:PGLS]; [A2MG:KPNA3:PGLS]; [HA:TIMP1:CA1]; [HA:TIMP1:KPNA3]; [HA:TIMP1:PGLS]; [HA:CA1:KPNA3]; [HA:CA1:PGLS]; [HA:KPNA3:PGLS]; [TIMPi:CA1:KPNA3]; [TIMPi:CA1:PGLS]; [TIMPi:KPNA3:PGLS]; [CA1:KPNA3:PGLS]; [A2MG:HA:TIMP1:CA1]; [A2MG:HA:TIMP1:KPNA3]; [A2MG:HA:TIMP1:PGLS]; [A2MG:HA:CA1:KPNA3]; [A2MG:HA:CA1:PGLS]; [A2MG:HA:KPNA3:PGLS]; [A2MG:TIMP1:CA1:KPNA3]; [A2MG:TIMP1:CA1:PGLS]; [A2MG:TIMP1:KPNA3:PGLS]; [A2MG:CA1:KPNA3:PGLS]; [HA:TIMP1:CA1:KPNA3]; [HA:TIMP1:CA1:PGLS]; [HA:TIMP1:KPNA3:PGLS]; [HA:CA1:KPNA3:PGLS]; [TIMPi:CA1:KPNA3:PGLS]; [A2MG:HA:TIMP1:CA1:KPNA3]; [A2MG:HA:TIMP1:CA1:PGLS]; [A2MG:HA:TIMP1:KPNA3:PGLS]; [A2MG:HA:CA1:KPNA3:PGLS]; [A2MG:TIMP1:CA1:KPNA3:PGLS]; [HA:TIMP1:CA1:KPNA3:PGLS]; and [A2MG:HA:TIMP1:CA1:KPNA3:PGLS].
6. The method of any one of embodiments 3 to 5, wherein a biomarker value is determined for 1, 2 or 3 biomarkers selected from A2MG, HA, and TIMP1 and the indicator is determined using the biomarker value(s).
7. The method of any one of embodiments 3 to 5, wherein a biomarker value is determined for 1, 2 or 3 biomarkers selected from CA1, KPNA3, and PGLS and the indicator is determined using the biomarker value(s).
8. The method of any one of embodiments 3 to 5, wherein biomarker values are determined for 2, 3, 4, 5 or 6 3 biomarkers selected from A2MG, HA, TIMP1, CA1, KPNA3, and PGLS and the indicator is determined using those biomarker values.
9. The method of any one of embodiments 3 to 5, wherein biomarker values are determined for each of A2MG, HA and TIMP1 and the indicator is determined using those biomarker values.
10. The method of any one of embodiments 3 to 5, wherein biomarker values are determined for each of CA1, KPNA3, and PGLS and the indicator is determined using those biomarker values.
11. The method of any one of embodiments 3 to 5, wherein biomarker values are determined for each of A2MG, HA, TIMP1, CA1, KPNA3, and PGLS and the indicator is determined using those biomarker values.
12. The method of any one of embodiments 1 to 11, wherein the subject is asymptomatic.
13. The method of any one of embodiments 1 to 11, wherein the subject has at least one clinical sign of liver fibrosis.
14. The method of any one of embodiments 1 to 11, wherein the subject has at least one clinical sign of liver cirrhosis.
15. The method of any one of embodiments 1 to 14, wherein the subject has a disease selected from hepatitis (e.g., a viral hepatitis such as Hepatitis A, Hepatitis B, Hepatitis C, Hepatitis D and Hepatitis E, or an autoimmune hepatitis), fatty liver disease (e.g., non-alcoholic fatty liver disease (NAFLD), non-alcoholic steatohepatitis (NASH), alcoholic fatty liver disease (AFLD) and alcoholic steatohepatitis (ASH)), alcoholic liver disease (ALD), primary sclerosing cholangitis (PSC), primary biliary cholangitis (PBC), hemochromatosis, Wilson's disease, drug-induced liver disease, and liver cancer (e.g., hepatocellular carcinoma), pediatric liver diseases that cause fibrosis and cirrhosis and all other recognized causes of liver fibrosis and cirrhosis.
16. The method of any one of embodiments 2 and 12 to 15, wherein the indicator indicates a likelihood of the presence or development of liver cirrhosis if:
   A2MG is present in the saliva sample at a higher level than in a reference saliva sample obtained from a healthy subject or from a subject with non-fibrotic liver disease;
   HA is present in the saliva sample at a higher level than in a reference saliva sample obtained from a healthy subject or from a subject with non-fibrotic liver disease; and
   TIMP1 is present in the saliva sample at a higher level than in a reference saliva sample obtained from a healthy subject or from a subject with non-fibrotic liver disease.
17. The method of any one of embodiments 2 and 12 to 15, wherein the indicator indicates a likelihood of the presence or development of liver cirrhosis if:
   A2MG is present in the saliva sample at a level corresponding to the level of A2MG in a reference saliva sample obtained from a subject with liver cirrhosis;
   HA is present in the saliva sample at a level corresponding to the level of HA in a reference saliva sample obtained from a subject with liver cirrhosis; and
   TIMP1 is present in the saliva sample at a level corresponding to the level of TIMP1 in a reference saliva sample obtained from a subject with liver cirrhosis.
18. The method of any one of embodiments 2 and 12 to 15, wherein the indicator indicates a likelihood of the absence of liver cirrhosis if:
   A2MG is present in the saliva sample at a level corresponding to the level of A2MG in a reference saliva sample obtained from a healthy subject or from a subject with non-fibrotic liver disease;
   HA is present in the saliva sample at a level corresponding to the level of HA in a reference saliva sample obtained from a healthy subject or from a subject with non-fibrotic liver disease; and
   TIMP1 is present in the saliva sample at a level corresponding to the level of TIMP1 in a reference saliva sample obtained from a healthy subject or from a subject with non-fibrotic liver disease.
19. The method of any one of embodiments 2 and 12 to 15, wherein the indicator indicates a likelihood of the absence of liver cirrhosis if:
   A2MG is present in the saliva sample at a level corresponding to the level of A2MG in a reference saliva sample obtained from a healthy subject or from a subject with non-fibrotic liver disease;
   HA is present in the saliva sample at a level corresponding to the level of HA in a reference saliva sample obtained from a healthy subject or from a subject with non-fibrotic liver disease; and
   TIMP1 is present in the saliva sample at a level corresponding to the level of TIMP1 in a reference saliva sample obtained from a healthy subject or from a subject with non-fibrotic liver disease.
20. The method of any one of embodiments 4 to 15, wherein the indicator indicates a likelihood of the presence or development of liver fibrosis or liver cirrhosis if:
   A2MG is present in the saliva sample at a higher level than in a reference saliva sample obtained from a healthy subject or from a subject with non-fibrotic liver disease;
   HA is present in the saliva sample at a higher level than in a reference saliva sample obtained from a healthy subject or from a subject with non-fibrotic liver disease;
   TIMP1 is present in the saliva sample at a higher level than in a reference saliva sample obtained from a healthy subject or from a subject with non-fibrotic liver disease;
   CA1 is present in the saliva sample at a higher level than in a reference saliva sample obtained from a healthy subject or from a subject with non-fibrotic liver disease;
   KPNA3 is present in the saliva sample at a lower level than in a reference saliva sample obtained from a healthy subject or from a subject with non-fibrotic liver disease; and/or
   PGLS is present in the saliva sample at a higher level than in a reference saliva sample obtained from a healthy subject or from a subject with non-fibrotic liver disease.
21. The method of any one of embodiments 4 to 15, wherein the indicator indicates a likelihood of the presence or development of liver fibrosis or liver cirrhosis if:
   A2MG is present in the saliva sample at a level corresponding to the level of A2MG in a reference saliva sample obtained from a subject with liver fibrosis or liver cirrhosis;
   HA is present in the saliva sample at a level corresponding to the level of HA in a reference saliva sample obtained from a subject with liver fibrosis or liver cirrhosis;
   TIMP1 is present in the saliva sample at a level corresponding to the level of TIMP1 in a reference saliva sample obtained from a subject with liver fibrosis or liver cirrhosis;
   CA1 is present in the saliva sample at a level corresponding to the level of CA1 in a reference saliva sample obtained from a subject with liver fibrosis or liver cirrhosis;
   KPNA3 is present in the saliva sample at a level corresponding to the level of KPNA3 in a reference saliva sample obtained from a subject with liver fibrosis or liver cirrhosis; and/or
   PGLS is present in the saliva sample a level corresponding to the level of PGLS in a reference saliva sample obtained from a subject with liver fibrosis or liver cirrhosis.
22. The method of any one of embodiments 4 to 15, wherein the indicator indicates a likelihood of the absence of liver fibrosis or liver cirrhosis if:
   A2MG is present in the saliva sample at a lower level than in a reference saliva sample obtained from a subject with liver fibrosis or liver cirrhosis;
   HA is present in the saliva sample at a lower level than in a reference saliva sample obtained from a subject with liver fibrosis or liver cirrhosis;
   TIMP1 is present in the saliva sample at a lower level than in a reference saliva sample obtained from a subject with liver fibrosis or liver cirrhosis;
   CA1 is present in the saliva sample at a lower level than in a reference saliva sample obtained from a subject with liver fibrosis or liver cirrhosis;
   KPNA3 is present in the saliva sample at a higher level than in a reference saliva sample obtained from a subject with liver fibrosis or liver cirrhosis; and/or
   PGLS is present in the saliva sample at a lower level than in a reference saliva sample obtained from a subject with liver fibrosis or liver cirrhosis.
23. The method of any one of embodiments 4 to 15, wherein the indicator indicates a likelihood of the absence of liver fibrosis or liver cirrhosis if:
   A2MG is present in the saliva sample at a level corresponding to the level of A2MG in a reference saliva sample obtained from a healthy subject or from a subject with non-fibrotic liver disease;
   HA is present in the saliva sample at a level corresponding to the level of HA in a reference saliva sample obtained from a healthy subject or from a subject with non-fibrotic liver disease;
   TIMP1 is present in the saliva sample at a level corresponding to the level of TIMP1 in a reference saliva sample obtained from a healthy subject or from a subject with non-fibrotic liver disease
   CA1 is present in the saliva sample at a level corresponding to the level of CA1 in a reference saliva sample obtained from a healthy subject or from a subject with non-fibrotic liver disease;
KPNA3 is present in the saliva sample at a level corresponding to the level of KPNA3 in a reference saliva sample obtained from a healthy subject or from a subject with non-fibrotic liver disease; and/or
   PGLS is present in the saliva sample at a level corresponding to the level of PGLS in a reference saliva sample obtained from a healthy subject or from a subject with non-fibrotic liver disease.
24. The method of any one of embodiments 1 to 23, further comprising applying a function to biomarker values to yield at least one functionalized biomarker value and determining the indicator using the at least one functionalized biomarker value.
25. The method of embodiment 24, wherein the function includes at least one of: (a) multiplying biomarker values; (b) dividing biomarker values; (c) adding biomarker values; (d) subtracting biomarker values; (e) a weighted sum of biomarker values; (f) a log sum of biomarker values; (g) a geometric mean of biomarker values; (h) a sigmoidal function of biomarker values; and (i) normalization of biomarker values.
26. The method of any one of embodiments 1 to 25, further comprising combining the biomarker values to provide a composite score and determining the indicator using the composite score.
27. The method of embodiment 26, wherein the biomarker values are combined by adding, multiplying, subtracting, and/or dividing biomarker values.
28. The method of any one of embodiments 1 to 27, further comprising analyzing the biomarker value(s), functionalized biomarker value(s) or composite score with reference to a corresponding reference biomarker value range or cut-off values, functionalized biomarker value range or cut-off values, or reference composite score range or cut-off values, to determine the indicator.
29. The method of embodiment 28, wherein the indicator indicates a likelihood of a presence or degree or severity of liver fibrosis if the biomarker value(s) or composite score is indicative of the levels of the biomarkers in the sample that correlate with an increased likelihood of a presence or degree or severity of liver fibrosis relative to a predetermined reference biomarker value range or cut-off value.
30. The method of embodiment 29, wherein the indicator indicates a likelihood of a presence or development of liver cirrhosis if the biomarker value(s) or composite score is indicative of the levels of the biomarkers in the sample that correlate with an increased likelihood of a presence or development of liver cirrhosis relative to a predetermined reference biomarker value range or cut-off value.
31. A method for monitoring liver fibrosis status or treatment of a subject, the method comprising, consisting or consisting essentially of:
   (1) determining a biomarker value for each of a plurality of biomarkers in a first saliva sample obtained from the subject, wherein a respective biomarker value is indicative of a level of a corresponding biomarker in the sample, and wherein the plurality of biomarkers comprises, consists or consists essentially of α-2-macroglobulin (A2MG), hyaluronic acid (HA), and tissue inhibitor matrix metalloproteinase 1 (TIMP1);
   (2) determining a first indicator using the biomarker values;
   (3) determining a biomarker value for each of the plurality of biomarkers in a second saliva sample obtained from the subject, wherein a respective biomarker value is indicative of a level of a corresponding biomarker in the second sample;
   (4) determining a second indicator using the biomarker values; and
   (5) comparing the first indicator with the second indicator, thereby monitoring the liver fibrosis status or treatment of the subject.
32. A method for monitoring liver fibrosis status or treatment of a subject, the method comprising, consisting or consisting essentially of:
   (1) determining a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from A2MG, HA, TIMP1, CA1, KPNA3 and PGLS;
   (2) determining a first indicator using the biomarker value(s);
   (3) determining a biomarker value for each of the at least one biomarkers, for which biomarker values were determined in the first saliva sample, in a second saliva sample obtained from the subject, wherein a respective biomarker value is indicative of a level of a corresponding biomarker in the second sample;
   (4) determining a second indicator using the biomarker value(s); and
   (5) comparing the first indicator with the second indicator, thereby monitoring the liver fibrosis status or treatment of the subject.
33. The method of embodiment 31 or embodiment 32, wherein the second indicator indicates reduced liver fibrosis relative to the liver fibrosis indicated by the first indicator, which is indicative of improved liver fibrosis status or effective treatment of the subject.
34. The method of embodiment 31 or embodiment 32, wherein the second indicator indicates unchanged liver fibrosis relative to the liver fibrosis indicated by the first indicator, which is indicative of an unchanged liver fibrosis status or a treatment that is effective in slowing progression of disease of the subject.
35. The method of embodiment 31 or embodiment 32, wherein the second indicator indicates increased liver fibrosis relative to the liver fibrosis indicated by the first indicator, which is indicative of worsening liver fibrosis status or an ineffective treatment of the subject.
36. The method of any one of embodiments 31 to 35, wherein the first sample is obtained from the subject before undergoing a therapeutic regimen for treating liver fibrosis and the second sample is obtained from the subject after undergoing the therapeutic regimen.
37. An apparatus for determining an indicator used in assessing a likelihood of a subject having a presence, absence or degree or severity of liver fibrosis, the apparatus comprising at least one electronic processing device that:
   determines a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of α-2-macroglobulin (A2MG), hyaluronic acid (HA), and tissue inhibitor matrix metalloproteinase 1 (TIMP1); and
   determines the indicator using the derived biomarker values.
38. An apparatus for determining an indicator used in assessing a likelihood of a subject having a presence, absence or development of liver cirrhosis, the apparatus comprising at least one electronic processing device that:
   determines a biomarker value for each of a plurality of biomarkers in a saliva sample obtained from the subject, wherein a respective biomarker value is indicative of a level of a corresponding biomarker in the sample, and wherein the plurality of biomarkers comprises, consists or consists essentially of α-2-macroglobulin (A2MG), hyaluronic acid (HA), and tissue inhibitor matrix metalloproteinase 1 (TIMP1); and
   determines the indicator using the derived biomarker values.
39An apparatus for determining an indicator used in assessing a likelihood of a subject having a presence, absence or degree or severity of liver fibrosis, the apparatus comprising at least one electronic processing device that:
   determines a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS); and
   determines the indicator using the derived biomarker value(s).
40. An apparatus for determining an indicator used in assessing a likelihood that liver cirrhosis is present, absent or developing in a subject, the apparatus comprising at least one electronic processing device that:
   determines a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS); and
   determines the indicator using the derived biomarker value(s).
41. A composition comprising a mixture of a saliva sample obtained from the subject, and for each of a plurality of biomarkers an antibody or antigen-binding fragment that binds specifically to the biomarker, wherein the plurality of biomarkers comprises, consists or consists essentially of α-2-macroglobulin (A2MG), hyaluronic acid (HA), and tissue inhibitor matrix metalloproteinase 1 (TIMP1).
42. A composition comprising a mixture of a saliva sample obtained from a subject, and for at least one (e.g., 1, 2, 3, 4, 5, 6, etc.) biomarker an antibody or antigen-binding fragment that binds specifically to the biomarker, wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS).
43. The composition of embodiment 42, wherein individual antibodies or antigen-binding fragments are labeled.
44. The composition of embodiment 43, wherein the composition comprises a plurality of antibodies or antigen-binding fragments, each of which specifically binds to a different biomarker and is associated with the same label or a different label, as compared to the biomarker specificity and associated label of other antibodies or antigen-binding fragments of the composition.
45. The composition of embodiment 44, wherein the labels associated with the different antibodies or antigen-binding fragments are detectably distinct.
46. A method for inhibiting the development or progression of liver fibrosis in a subject, the method comprising exposing the subject to a treatment regimen for treating liver fibrosis at least in part on the basis that the subject is determined by the indicator-determining method of any one of embodiments 1 to 36 as having a likelihood of a presence or degree or severity of liver fibrosis.
47. A method for inhibiting the development or progression of liver cirrhosis in a subject, the method comprising exposing the subject to a treatment regimen for treating liver cirrhosis at least in part on the basis that the subject is determined by the indicator-determining method of any one of embodiments 2 to 36 as having a likelihood of a presence or development of liver cirrhosis.
48. The method of embodiment 46 or embodiment 47, wherein the subject has been administered a treatment regimen prior to undertaking the indicator-determining method.
49. The method of embodiment 46 or embodiment 47, wherein the subject has not undergone a treatment regimen prior to undertaking the indicator-determining method.
50. The method of any one of embodiments 46, 48 and 49, further comprising: taking a sample from the subject and determining an indicator indicative of a likelihood of a presence or degree or severity of liver fibrosis using the indicator-determining method.
51. The method of any one of embodiments 47 to 49, further comprising: taking a sample from the subject and determining an indicator indicative of a likelihood of a presence or degree or severity of liver cirrhosis using the indicator-determining method.
52. The method of any one of embodiments 46 to 51, further comprising sending a sample obtained from the subject to a laboratory at which the indicator is determined according to the indicator-determining method, and optionally receiving the indicator from the laboratory.
53. The method of embodiment 52, further comprising: receiving the indicator from the laboratory.
54. A kit for determining an indicator used in assessing a likelihood of a subject having a presence, absence or degree or severity of liver fibrosis, or a likelihood that liver cirrhosis is present, absent or developing in a subject, the kit comprising: for each of a plurality of biomarkers an antibody or antigen-binding fragment that binds specifically to the biomarker, wherein the plurality of biomarkers comprises, consists or consists essentially of α-2-macroglobulin (A2MG), hyaluronic acid (HA), and tissue inhibitor matrix metalloproteinase 1 (TIMP1).
55. A kit for determining an indicator used in assessing a likelihood of a subject having a presence, absence or degree or severity of liver fibrosis, or a likelihood that liver cirrhosis is present, absent or developing in a subject, the kit comprising: for at least one biomarker an antibody or antigen-binding fragment that binds specifically to the biomarker, wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS).
56. The kit of embodiment 54 or embodiment 55, further comprising at least one reagent for preparing a saliva sample for biomarker (e.g., protein biomarker and/or polysaccharide biomarker) analysis.
57. The kit of any one of embodiments 54 to 56, further comprising one or more of buffer(s), positive and negative controls, and reaction vessel(s).
58. The kit of any one of embodiments 54 to 57, further comprising instructions for performing the indicator-determining method of any one of embodiments 1 to 36.

In order that the disclosure may be readily understood and put into practical effect, particular preferred embodiments will now be described by way of the following non-limiting example.

### EXAMPLES

### Example 1

### Diagnostic Utility of Salivary Biomarkers for Diagnosis of Liver Cirrhosis

### Characteristics of Participants

The clinical characteristics of patients included in the training (n=40) and validation (n=95) sets are statistically similar (Table 1). The average age of participants in the training (60±9) and validation (64±7) was statistically similar, and gender distribution was consistent between the groups (57% male in the training vs 57% male in the validation set). Three main causes of liver disease were identified in the populations: NAFLD (training: 76.6%, validation: 82.8%), alcohol (training: 3.3%, validation: 3.7%), and viral hepatitis (training: 6.7%, validation: 6.1%). These etiologies were evenly distributed between the cohorts. Regarding the stage of fibrosis measured using TE, the training cohort had a higher proportion of patients with intermediate degrees of liver fibrosis (25.0% in the training cohort vs 10.6% in the validation), which was predominantly caused by the number of patients in each group. No differences were observed between the training and validation sets regarding the clinical parameters and liver enzymes.

TABLE 1
Characteristics of participants in the training and validation sets.

| Characteristic | Training set | | Validation set | |
|---|---|---|---|---|
| n | 40 | | 95 | |
| Male, % | 23 | (57) | 55 | (57) |
| Age, years | 60 | (9) | 64 | (7) |
| AST, IU/L | 40 | (22) | 52 | (20) |
| ALT, IU/L | 50 | (18) | 58 | (24) |
| AP, IU/L | 88 | (29) | 78 | (14) |
| Albumin, g/dL | 36 | (4) | 39 | (6) |
| Cause of liver disease | | | | |
| NAFLD, % | 23 | (76.6) | 67 | (82.8) |
| Alcohol, % | 1 | (3.3) | 3 | (3.7) |
| Viral hepatitis, % | 2 | (6.7) | 5 | (6.1) |
| Other, % | 4 | (1.4) | 6 | (7.4) |
| Fibrosis stage | | | | |
| % of patients without fibrosis | 20 | (50.0%) | 54 | (56.8) |
| % of patients with intermediate fibrosis | 10 | (25.0) | 10 | (10.6) |
| % of patients with liver cirrhosis | 10 | (25.0) | 31 | (32.6) |

Data are expressed as means (standard deviation) or number (proportion)
AST: aspartate aminotransferase,
ALT: alanine aminotransferase,
AP: alkaline phosphatase,
NAFLD: non-alcoholic fatty liver disease

### Biomarker Quantification in Serum and Saliva Samples

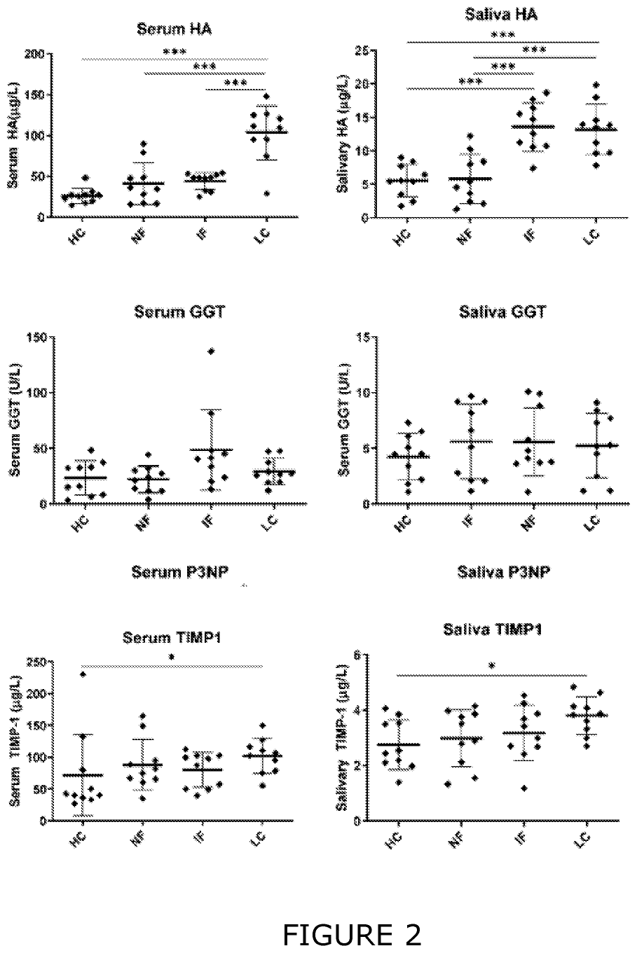

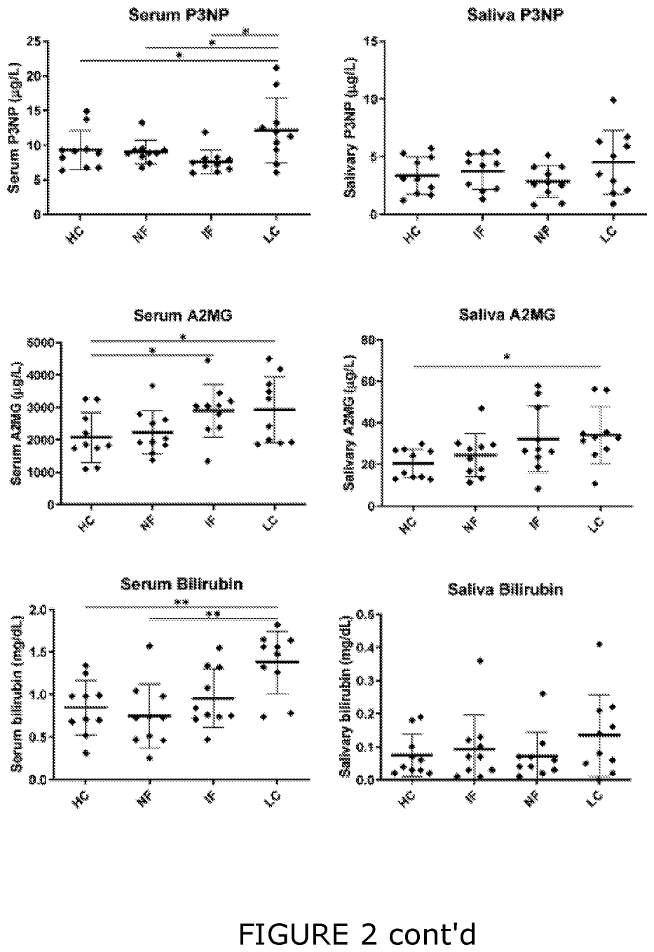

Six serum biomarkers currently used for the detection of liver cirrhosis were measured in paired serum and saliva samples. For HA, TIMP-1, P3NP and A2MG, the concentrations were measured using ELISA. Spike and recovery tests were performed to validate the use of those commercial kits with saliva samples. Significantly higher mean concentrations (p<0.05) of HA, A2MG, P3NP, and total bilirubin were detected in the serum of LC patients compared to the controls (FIG. 2, Table 2). Patients in the IF cohort also showed significantly higher mean concentrations of A2MG (p<0.05) compared to those in the NF cohort and controls. For TIMP-1, the mean serum concentration was significantly increased (p<0.05) in LC patients compared to the controls, but no differences were observed between the LC and IF cohorts.

All six biomarkers were successfully detected in saliva samples, but at lower concentrations compared to serum, which was anticipated given the sample matrix. Mean concentrations of salivary HA, TIMP-1, and A2MG were higher in patients with LC (p<0.05) compared to HC. Furthermore, a significant increase (p<0.0001) in the mean concentration of HA in saliva was also observed in patients in the IF cohort compared to HC and NF (FIG. 2, Table 2). No significant differences were observed between the groups in either serum or salivary GGT levels, and the increase in total bilirubin observed in the serum samples was not observed in saliva. The Spearman's Rho correlation showed a significant positive correlation between the serum and salivary concentrations of HA (r=0.546, p<0.01), A2MG (r=0.326, p<0.05), and total bilirubin (r=0.482, p<0.05).

TABLE 2
Quantification of liver fibrosis biomarkers in serum and saliva of
patients with live cirrhosis, intermediate degrees of fibrosis, non-
fibrotic liver conditions and healthy controls in the training set.

| | HC | | NF | | IF | | LC | | p-value |
|---|---|---|---|---|---|---|---|---|---|
| Age, years | 65 | (8) | 59 | (10) | 64 | (9) | 61 | (10) | 0.3054 |
| LSM, kPa | 5.3 | (1.4) | 5 | (1.2) | 9.8 | (1.4) | 22.6 | (4) | <0.0001 |
| Serum HA, μg/L | 26.2 | (9.3) | 41.4 | (25.8) | 44.2 | (10.4) | 103.5 | (33) | <0.0001 |
| Saliva HA, μg/L | 5.6 | (2.4) | 5.8 | (3.7) | 13.6 | (3.6) | 13.2 | (3.8) | <0.0001 |
| Serum TIMP-1, μg/L | 71.5 | (64.1) | 88.2 | (40.1) | 79.9 | (27.5) | 101.7 | (27.3) | 0.4414 |
| Saliva TIMP-1, μg/L | 2.8 | (0.9) | 3 | (1) | 3.2 | (1) | 3.8 | (0.7) | 0.0847 |
| Serum A2MG, mg/L | 2.1 | (0.8) | 2.2 | (0.7) | 2.9 | (0.8) | 2.9 | (1) | 0.0488 |
| Saliva A2MG, μg/L | 20.4 | (7) | 24.5 | (10.5) | 32.2 | (15.9) | 34.2 | (13.6) | 0.0475 |
| Serum P3NP, μg/L | 9.3 | (2.9) | 9 | (1.7) | 7.6 | (1.7) | 12.2 | (4.7) | 0.0144 |
| Saliva P3NP, μg/L | 3.4 | (1.6) | 2.9 | (1.4) | 3.7 | (1.5) | 4.5 | (2.8) | 0.2803 |
| Serum bilirubin, mg/L | 0.85 | (0.32) | 0.75 | (0.38) | 0.96 | (0.35) | 1.38 | (0.36) | 0.0016 |
| Saliva bilirubin, mg/dL | 0.07 | (0.06) | 0.07 | (0.07) | 0.09 | (0.1) | 0.14 | (0.12) | 0.4087 |
| Serum GGT, IU/L | 23.4 | (15.4) | 22.1 | (12) | 48.5 | (36.4) | 29.3 | (11.7) | 0.0331 |
| Saliva GGT, IU/L | 4.3 | (2.1) | 5.6 | (3) | 5.6 | (3.4) | 5.2 | (2.9) | 0.6953 |
| ELF score | 6.2 | (0.4) | 7.7 | (0.6) | 8.7 | (0.4) | 9.8 | (0.5) | <0.0001 |
| Hepascore | 0.1 | (0.1) | 0.2 | (0.2) | 0.4 | (0.2) | 0.7 | (0.3) | <0.0001 |

| | | | | | | | | |
|---|---|---|---|---|---|---|---|---|
| FIB-4 | N/A | 1.1 | (0.4) | 1.8 | (0.3) | 3.9 | (1.6) | <0.0001 |
| APRI | N/A | 0.5 | (0.2) | 0.8 | (0.3) | 1.9 | (0.5) | <0.0001 |

Data are expressed as means (standard deviation)
LSM: liver stiffness measurement, ELF: Enhanced Liver Fibrosis score, FIB-4: Fibrosis-4, APRI: AST to Platelet ratio index. Statistical significance determined using ANOVA.

### Development of the Saliva Liver Fibrosis (SALF) Score

The diagnostic performance of the biomarkers to predict liver cirrhosis was assessed using receiver-operating characteristic (ROC) curve analysis. Overall, the highest areas under the ROC curve (AUCs) were obtained when liver cirrhosis patients were compared to healthy controls. In serum, the biomarkers with the highest AUC for the identification of LC against HC were HA, total bilirubin, and TIMP-1 with AUC values of 0.980, 0.840, and 0.750, respectively (Table 3). HA, A2MG, and TIMP-1 showed the best performance in saliva samples with AUCs of 0.971, 0.850, and 0.830, respectively. When compared to the measurements in serum, the salivary biomarkers presented slightly lower AUCs with the exception of TIMP-1, in which the performance in saliva was superior (salivary AUC: 0.830 vs serum AUC: 0.750) (Table 3). Furthermore, the concentrations of these 3 biomarkers in saliva were independently associated with the degree of liver fibrosis as observed by a significant positive correlation between LSM and HA (r=0.474, p<0.001), TIMP-1 (r=0.202, p=0.046), and A2MG (r=0.389, p<0.001) levels.

TABLE 3
Area Under the Curve (AUC) for serum and saliva biomarkers
in liver cirrhosis patients compared to patients with
non-fibrotic liver conditions and healthy individuals.

| | | | | | |
|---|---|---|---|---|---|
| Variable | AUC | Sens | Spec | PPV | NPV |

LC vs HC

| | | | | | |
|---|---|---|---|---|---|
| Serum HA | 0.980 | 90 | 100 | 90.9 | 100 |
| Saliva HA | 0.971 | 90 | 100 | 90.9 | 100 |
| Serum TIMP1 | 0.750 | 100 | 6 | 100 | 71.4 |
| Saliva TIMP-1 | 0.830 | 100 | 60 | 100 | 71.4 |
| Serum A2MG | 0.730 | 90 | 60 | 85.7 | 69.2 |
| Saliva A2MG | 0.850 | 80 | 90 | 81.8 | 88.9 |
| Serum GGT | 0.590 | 90 | 50 | 83.3 | 64.3 |
| Saliva GGT | 0.635 | 40 | 90 | 60 | 80 |
| Serum bilirubin | 0.840 | 80 | 90 | 81.8 | 88.9 |
| Saliva bilirubin | 0.645 | 80 | 50 | 71.4 | 61.5 |
| Serum P3NP | 0.700 | 80 | 70 | 77.8 | 73.7 |
| Saliva P3NP | 0.640 | 40 | 100 | 62.5 | 100 |

LC vs NF

| | | | | | |
|---|---|---|---|---|---|
| Serum HA | 0.920 | 80 | 100 | 83.3 | 100 |
| Saliva HA | 0.910 | 90 | 80 | 88.9 | 81.8 |
| Serum TIMP1 | 0.480 | 100 | 30 | 100 | 49.9 |
| Saliva TIMP-1 | 0.720 | 90 | 50 | 83.3 | 64.3 |
| Serum A2MG | 0.590 | 40 | 90 | 60 | 80 |
| Saliva A2MG | 0.750 | 70 | 90 | 75 | 87.5 |
| Serum GGT | 0.660 | 100 | 30 | 100 | 48.8 |
| Saliva GGT | 0.510 | 100 | 20 | 100 | 45.6 |
| Serum bilirubin | 0.885 | 80 | 90 | 81.8 | 88.9 |
| Saliva bilirubin | 0.675 | 60 | 80 | 64.7 | 75 |
| Serum P3NP | 0.750 | 80 | 80 | 80 | 80 |
| Saliva P3NP | 0.670 | 50 | 90 | 64.3 | 83.3 |

LC vs HC + NF

| | | | | | |
|---|---|---|---|---|---|
| Serum HA | 0.950 | 90 | 90 | 94.7 | 81.8 |
| Saliva HA | 0.945 | 90 | 90 | 94.7 | 81.8 |
| Serum TIMP1 | 0.635 | 100 | 35 | 100 | 43.5 |
| Saliva TIMP-1 | 0.775 | 90 | 55 | 91.7 | 50 |
| Serum A2MG | 0.660 | 90 | 45 | 90 | 45 |
| Saliva A2MG | 0.800 | 70 | 95 | 86.4 | 87.5 |
| Serum GGT | 0.625 | 90 | 45 | 90 | 45 |
| Saliva GGT | 0.562 | 60 | 65 | 76.5 | 46.2 |
| Serum bilirubin | 0.863 | 80 | 90 | 90 | 80 |
| Saliva bilirubin | 0.660 | 60 | 75 | 78.9 | 54.5 |
| Serum P3NP | 0.725 | 80 | 75 | 88.9 | 61.5 |
| Saliva P3NP | 0.655 | 40 | 100 | 76.9 | 100 |

LC + IF vs HC + NF

| | | | | | |
|---|---|---|---|---|---|
| Serum HA | 0.858 | 75 | 85 | 77.3 | 83.3 |
| Saliva HA | 0.948 | 90 | 90 | 90 | 90 |
| Serum TIMP1 | 0.603 | 55 | 75 | 62.5 | 68.8 |
| Saliva TIMP-1 | 0.682 | 90 | 45 | 81.8 | 62.1 |
| Serum A2MG | 0.755 | 60 | 85 | 68 | 80 |
| Saliva A2MG | 0.738 | 55 | 95 | 67.9 | 91.8 |
| Serum GGT | 0.703 | 90 | 45 | 81.8 | 62.5 |
| Saliva GGT | 0.560 | 45 | 80 | 59.3 | 69.2 |
| Serum bilirubin | 0.729 | 55 | 90 | 66.7 | 84.6 |
| Saliva bilirubin | 0.606 | 50 | 75 | 60 | 66.7 |
| Serum P3NP | 0.480 | 40 | 85 | 58.6 | 72.7 |
| Saliva P3NP | 0.642 | 55 | 75 | 62.5 | 68.5 |

Sens: Sensitivity (%);
Spec: Specificity (%);
PPV: Positive Predictive Value (%);
NPV: Negative Predictive Value (%)

The above data show that salivary HA, TIMP-1 and A2MG individually have good performance (AUC >0.800) for differentiating liver cirrhosis patients from healthy controls. By contrast, the performance of each of GGT, bilirubin and P3NP was suboptimal (AUC 0.635-0.645) with lower sensitivity/specificity.

Next, the salivary biomarkers assessed in the training set were used in a logistic regression analysis to create a diagnostic algorithm for liver cirrhosis. First, specific combinations of biomarkers were tested in an effort to improve the diagnostic performance of the proposed test (Table 4). The optimal biomarker panel was selected based on the highest AUC values, as well the highest Youden's index (3), calculated using J=(sensitivity+specificity)−1. The highest J was obtained by combining HA, TIMP-1 and A2MG (0.95+0.90-1=0.85), whereas the lowest J was observed for the combination of bilirubin, P3NP, and GGT (0.60+0.93-1=0.53).

TABLE 4
Receiver-operating characteristic (ROC)
analysis of potential biomarker panels.

| Biomarker panel | AUC | Sens (%) | Spec (%) |
|---|---|---|---|
| HA + TIMP1 + A2MG | 0.970 | 95.0 | 90.0 |
| TIMP1 + HA + GGT | 0.843 | 100.0 | 66.7 |
| HA + GGT + A2MG | 0.800 | 100.0 | 56.7 |
| HA + P3NP + GGT | 0.793 | 90.0 | 60.0 |
| Bilirubin + TIMP1 + GGT | 0.790 | 100.0 | 46.7 |
| A2MG + GGT + Bilirubin | 0.733 | 90.0 | 53.3 |
| Bilirubin + P3NP + GGT | 0.730 | 60.0 | 93.3 |

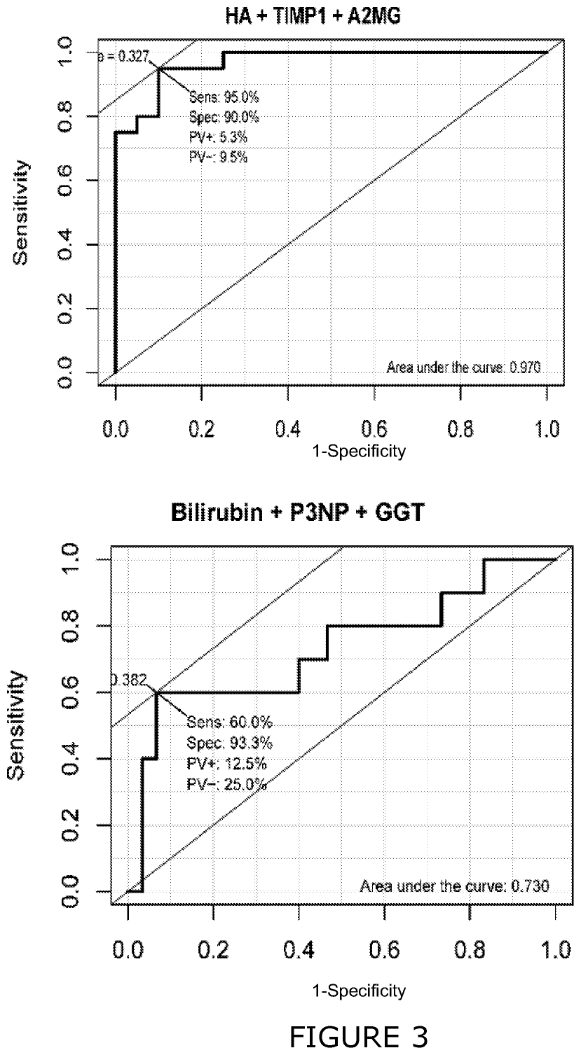

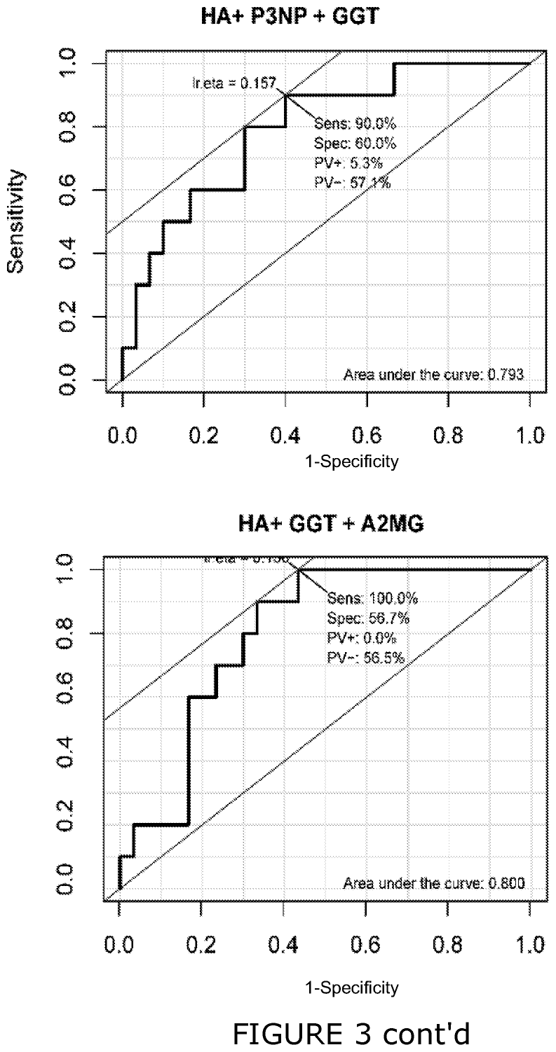

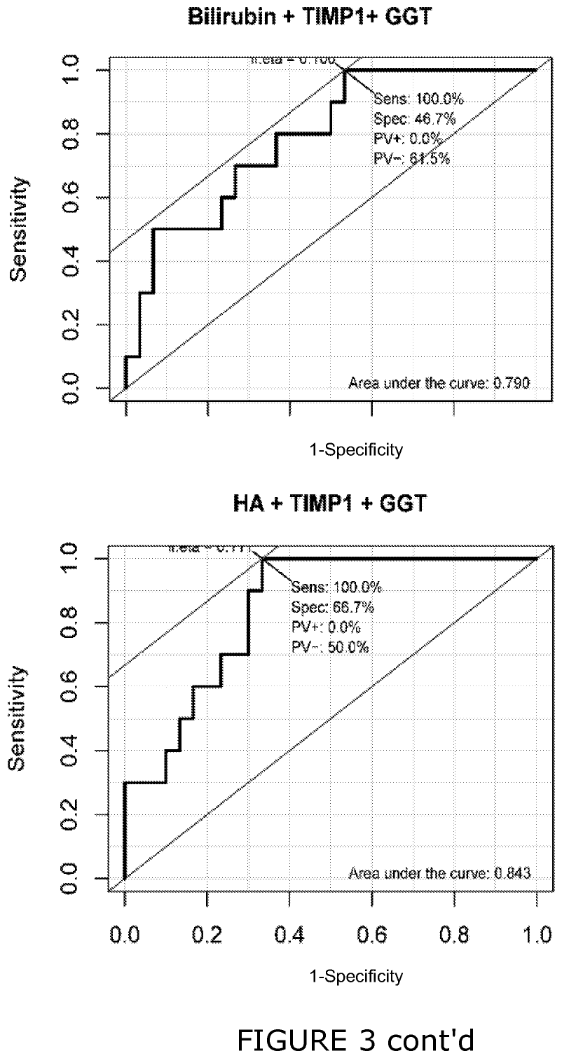

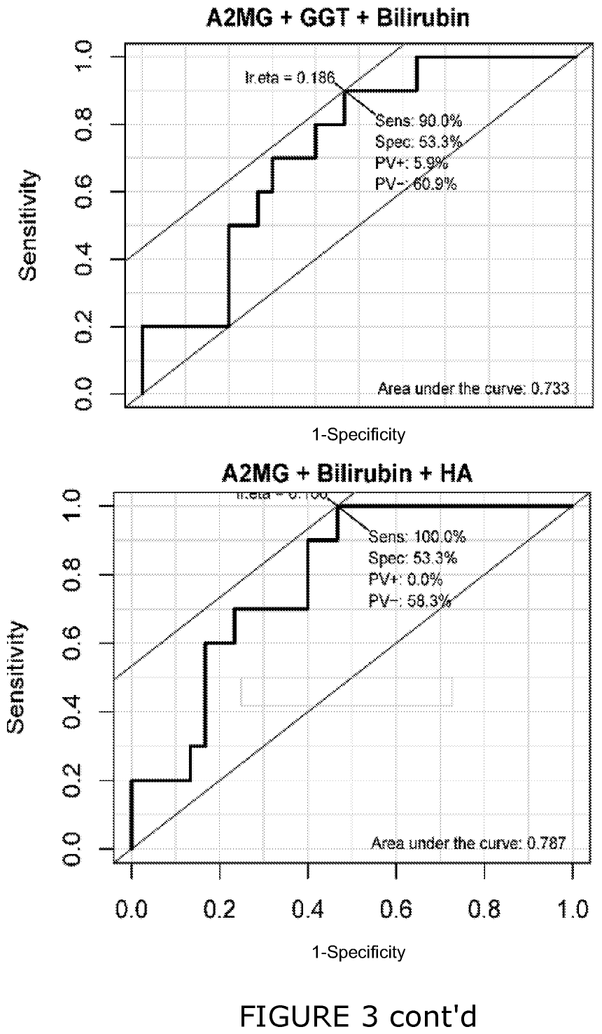

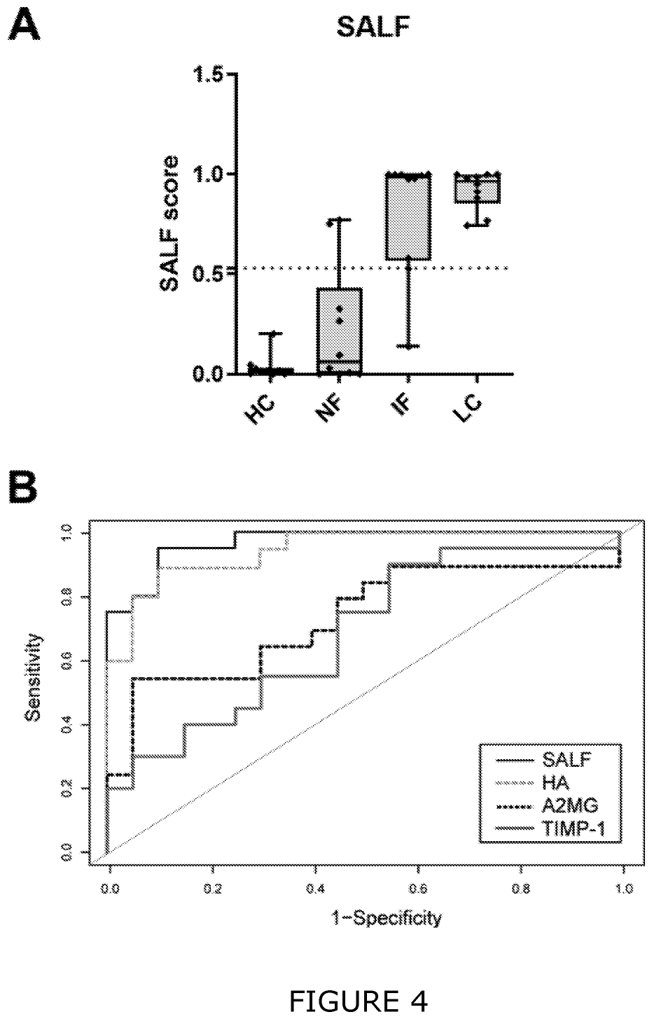

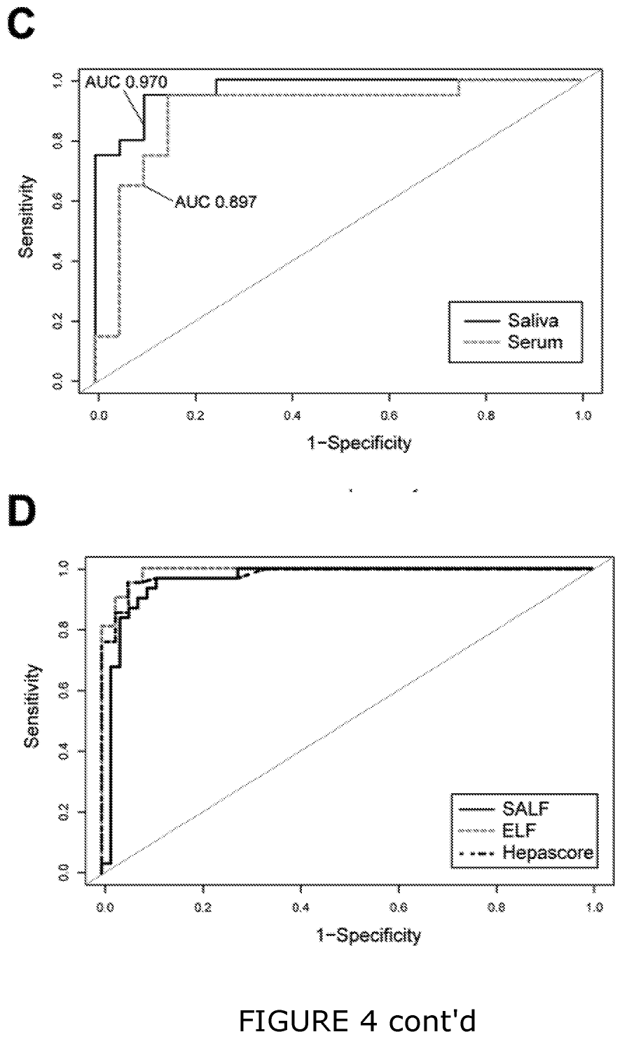

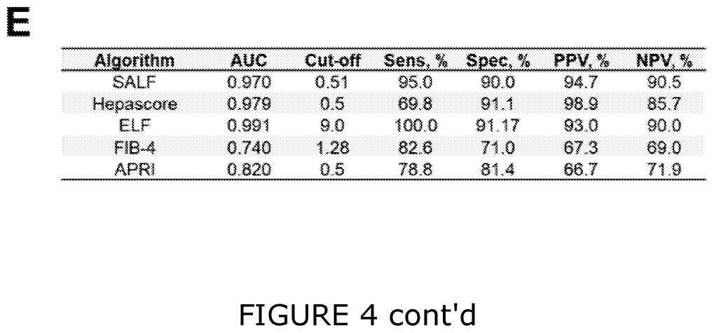

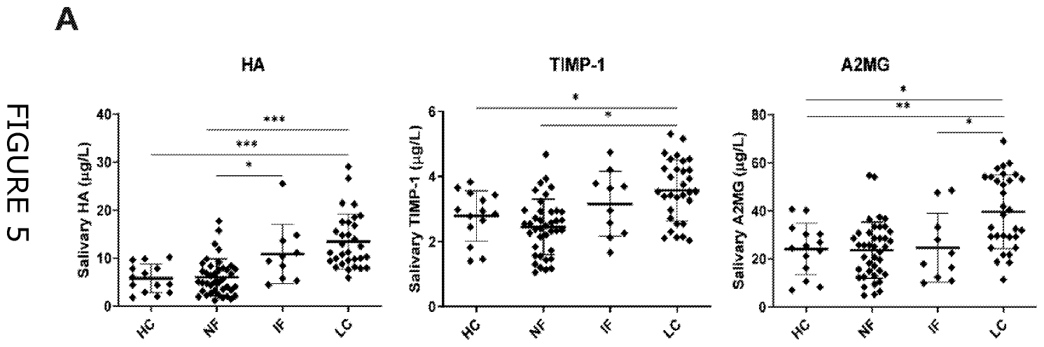

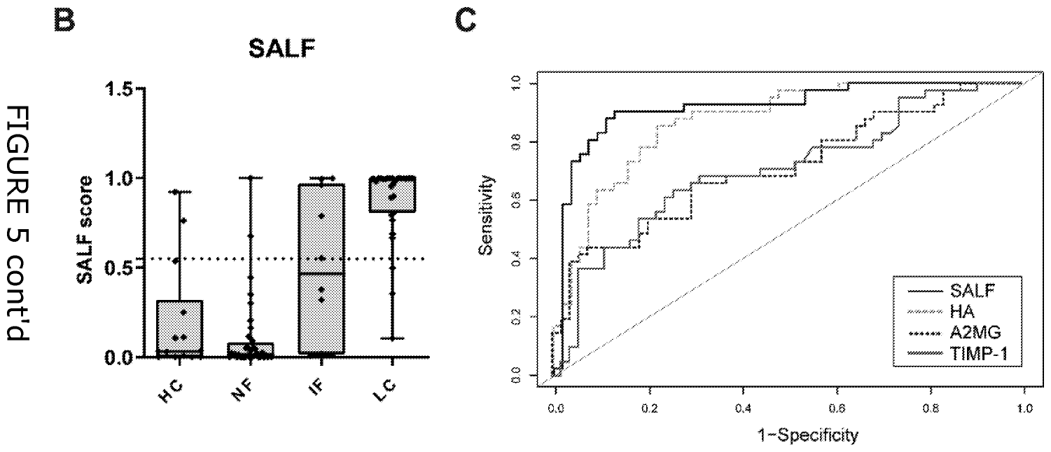

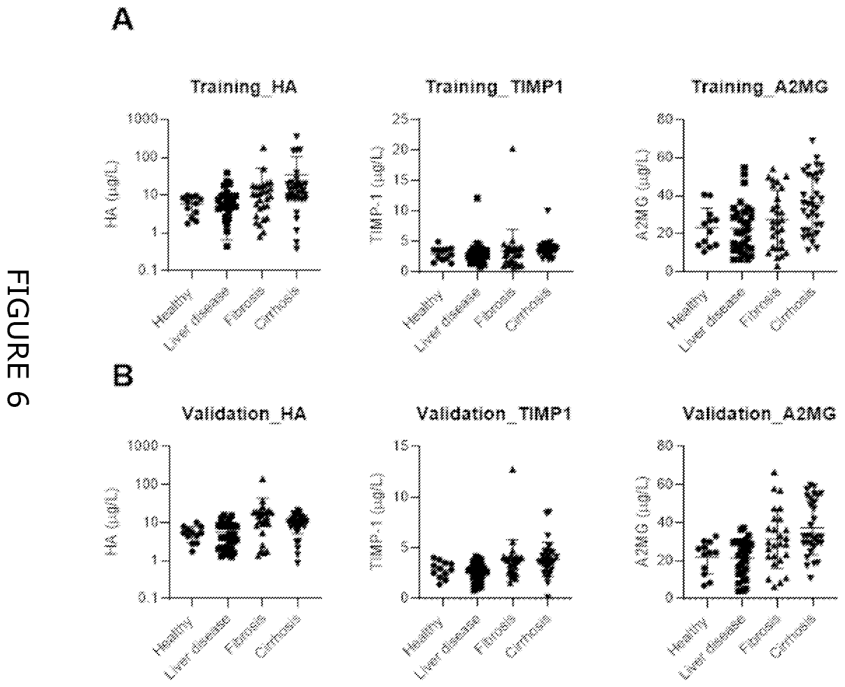

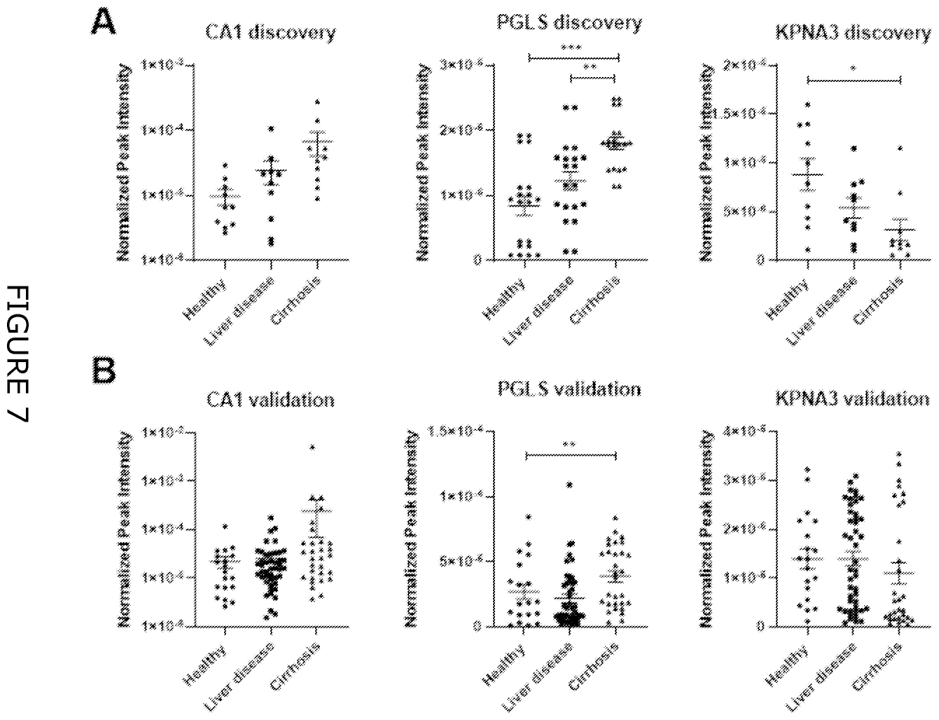

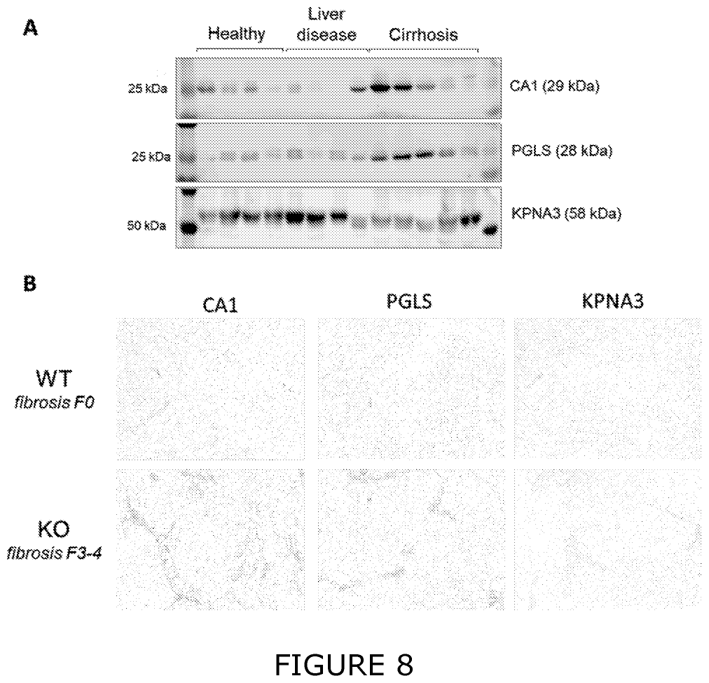

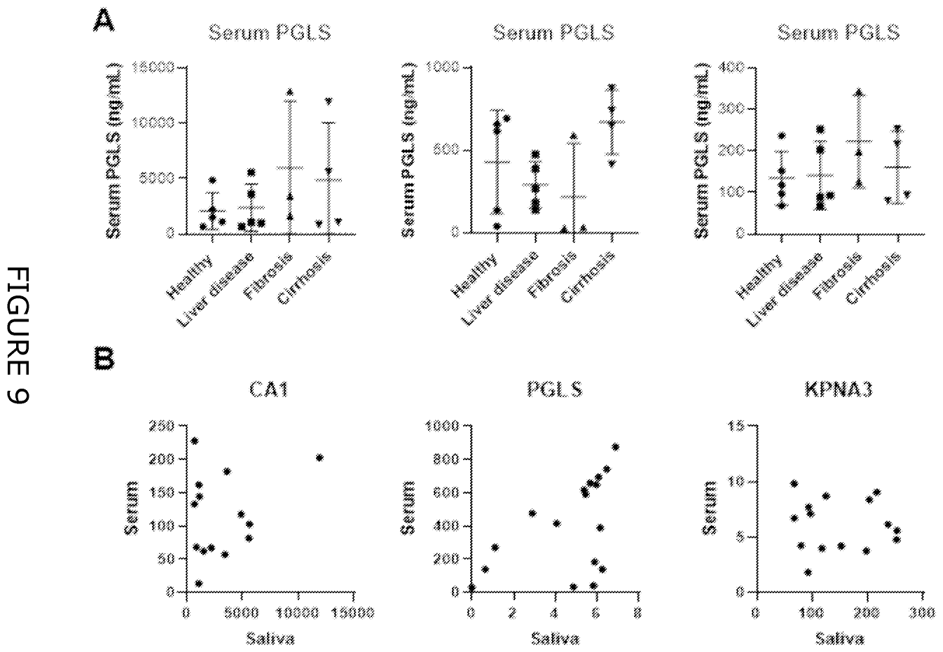

The analysis of individual ROC curves confirmed the superior performance of the panel containing HA, TIMP-1 and A2MG, with an AUC of 0.970 with a high sensitivity (95.0%/) and specificity (90.00/) (FIG. 3). Based on this panel, a Saliva Liver Fibrosis score (SALF) was developed for determining the likelihood of the presence or absence of liver cirrhosis. The SALF score was calculated using the following algorithm: SALF=(Y/Y+1), in which Y=EXP[—15.8454816+(0.7944629*HA)+(1.3469354*TIMP-1)+(0.1541859*A2MG)]. The SALF scores of LC patients (0.921±0.09) and IF patients (0.819±0.285) were significantly higher (p<0.0001) than the score of the healthy controls (0.034±0.05) and NF patients (0.061±0.29) (FIG. 4A). No significant differences in the SALF scores were observed between the LC and IF cohorts.

This model provided a high AUC of 0.990 for the detection of LC compared to the HC, with 100.0% sensitivity and 95.0% specificity (cut-off: 0.55). In the comparison between LC and NF patients, the SALF score had an AUC, sensitivity and specificity of 0.997, 100.0%, and 95.0%, respectively. For the detection of liver fibrosis (LC+IF combined vs HC+NF cohorts), the AUC was 0.970 (sensitivity 95.0% and specificity 90.0%). The SALF score showed a diagnostic performance which was significantly higher than its individual parameters for all the conditions tested (FIG. 4B). Furthermore, the combination of the same markers that compose the SALF score showed a better performance in saliva than in serum. The performance of the combination of HA, TIMP-1 and A2MG in serum had an AUC of 0.897 with a sensitivity of 95.0% and specificity of 85.0% when comparing liver fibrosis patients (LC+IF) vs HC+NF (FIG. 4C). The SALF score was compared to clinically validated serum algorithms to diagnose LC, in which the performance of the saliva score was superior to the FIB-4 (AUC: 0.740), and APRI (AUC: 0.820), and similar to the Hepascore (AUC: 0.979). The ELF score showed the best performance, with an AUC of 0.991, 100.0% sensitivity and 91.7% specificity for the detection of fibrosis (FIGS. 3D and 3E). A strong positive correlation was observed between the SALF score and the Hepascore (r=0.672, p<0.001) and ELF score (r=0.669, p<0.001), whereas a moderate correlation was observed for the APRI (r=0.354, p<0.05), and FIB-4 (r=0.388, p<0.005). To further refine the multi-marker panel, the bootstrap validation method was applied, with an average AUC, sensitivity, specificity, NPV, and PPV were 0.96, 84.9%, 91.7%, 77.3%, and 95.1%, respectively.

### Validation of the SALF Score

To further investigate the feasibility of the SALF score to be used as a diagnostic/screening tool, the performance of the algorithm was validated using an independent cohort of patients with different degrees of fibrosis: 14 healthy controls (HC), 40 patients with non-fibrotic liver conditions (NF), 10 patients with intermediate degrees of hepatic fibrosis (IF) and 31 liver cirrhosis patients (LC). In the validation set, the concentrations of HA, TIMP-1 and A2MG were significantly increased in the saliva of LC patients when compared to patients in the HC and NF cohort (p<0.05, FIG. 5A). Furthermore, the mean concentration of salivary HA was increased patients in the IF cohort compared to those in the NF (p<0.05). The SALF score for each patient was calculated according to the previous algorithm. The median SALF scores of the LC (0.88±0.21) patients was significantly higher (p<0.01) than in the HC (0.20±0.31) and NF cohorts (0.09±0.20). The IF cohort showed SALF scores (0.50±0.41) that were significantly different from LC (p<0.01), NF (p<0.01) and HC (p<0.05). No statistically significant differences were observed between HC and NF (p=0.563) (FIG. 5B). Using the optimal cut-off of 0.51, the SALF score showed an AUC of 0.962, with 87.1% sensitivity, 94.4% specificity, 92.7% PPV, and 90.0% NPV to detect LC patients against those without fibrosis (HC+NF). For the detection of liver fibrosis (LC+IF), the AUC, sensitivity, specificity, PPV and NPV were 0.920, 90.2%, 87.0%, 92.2% and 84.1%, respectively. Similar to the training set, the performance of the combinatorial algorithm was superior to the performance of its components individually (Table 5).

TABLE 5
Accuracy of liver fibrosis (LC + IF vs HC +
NF) in the training, validation, and total cohort
using the SALF score, in comparison to its constitutes.

| | | | | |
|---|---|---|---|---|
| AUC | Sens, % | Spec, % | PPV, % | NPV, % |

Training Cohort

| | | | | | |
|---|---|---|---|---|---|
| SALF | 0.970 | 95 | 90 | 94.7 | 90.5 |
| HA, μg/L | 0.948 | 90 | 90 | 90 | 90 |
| TIMP-1, μg/L | 0.682 | 90 | 45 | 81.8 | 62.1 |
| A2MG, μg/L | 0.738 | 55 | 95 | 67.9 | 91.8 |

Validation Cohort

| | | | | | |
|---|---|---|---|---|---|
| SALF | 0.920 | 90.2 | 87 | 92.2 | 84.1 |
| HA, μg/L | 0.873 | 85.4 | 77.8 | 87.5 | 74.5 |
| TIMP-1, μg/L | 0.703 | 63.4 | 74.1 | 72.7 | 65 |
| A2MG, μg/L | 0.711 | 43.9 | 92.6 | 68.5 | 81.8 |

Total cohort

| | | | | | |
|---|---|---|---|---|---|
| SALF | 0.936 | 90.2 | 89.2 | 91.7 | 87.3 |
| HA, μg/L | 0.894 | 78.7 | 86.5 | 83.1 | 82.8 |
| TIMP-1, μg/L | 0.706 | 70.5 | 664.9 | 72.7 | 62.3 |
| A2MG, μg/L | 0.716 | 54.1 | 83.8 | 68.9 | 73.3 |

### DISCUSSION

Despite the advances in the diagnosis of liver cirrhosis and the recommendation of early management of chronic liver disease by the European Association for the Study of the Liver (EASL), approximately two-thirds of liver cirrhosis patients are diagnosed in advanced stages (Asrani et al., 2019. J Hepatol 70(1):151-171; D'Amico et al., 2006. J Hepatol 44(1):217-231). Furthermore, the 5-year survival rates for patients with cirrhosis decrease from 67% to 45% once the disease reach the decompensated stage, in which clinical symptoms are perceived and extra-hepatic complications (e.g., variceal bleeding and ascites) are common (Asrani et al., 2019. supra). To allow for early clinical interventions and avoid the need for liver biopsy, several non-invasive approaches to detect liver fibrosis have been reported, such as blood fibrosis tests, liver elastography, and their combination (Boursier et al., 2017. J Hepatol 66(6):1158-1165). For instance, magnetic resonance elastography (MRE) showed a diagnostic accuracy of 97.1% to detect advanced liver fibrosis and 97.9% for cirrhosis (Shi et al., 2016. Am J Gastronterol 111(6):823-833). However, the current scenario presents two significant challenges. First, the estimated prevalence of non-alcoholic fatty liver disease (NALFD), the most common liver disease worldwide, is 25% in Caucasian populations (Younossi et al., 2018. Nat Rev Gastroenterol Hepatol (1):11-20). Approximately 21% to 26% of these patients progress to more severe conditions such as non-alcoholic steatohepatitis (NASH) and/or cirrhosis (Ahmed et al., 2015. J Gastroenterol Hepatol 13(12):2062-2070). Therefore, the screening of all NAFLD patients using elastography is not feasible due to the large number of individuals to be screened, as well as the high rate of unnecessary tests (Boursier et al., 2017. supra). The second challenge is to provide widely accessible tools for the screening and diagnosis of cirrhosis. Elastography techniques are only available in specialized tertiary centers or clinics, and the cost per examination is too high to allow for its use as a screening tool (Boursier et al., 2017. supra). Recent studies, however, highlight the increasing burden of chronic liver disease in indigenous, rural and regional communities especially linked to lower incomes and levels of education, restricted access to care, and older age of the population (Glenister et al., 2018. BMC Public Health 18(1):1-10; Roberts et al., 2021. Med J Aust). In this context, the development of a readily accessible, cost-effective screening test to identify patients who require close monitoring would significantly improve patient outcomes.

The present study was conducted to develop a simple saliva-based score for the detection of liver fibrosis, named Saliva Liver Fibrosis (SALF) score. We analyzed six serum markers for fibrosis—hyaluronic acid (HA), tissue inhibitor of metalloproteinase 1 (TIMP-1), α-2-macroglobulin (A2MG), γ-glutamyltransferase (GGT), total bilirubin, and the amino-terminal type III procollagen peptide (P3NP)—in serum and saliva samples from liver disease patients with different degrees of fibrosis and healthy volunteers. The SALF score accurately detected patients with liver cirrhosis within a population of healthy individuals and patients with underlying liver disease. Furthermore, the score can be further developed to be used for the early detection of liver fibrosis, as demonstrated by the performance of the novel algorithm in patients with intermediate degrees of fibrosis (F2 and F3).

These findings were subsequently validated in an independent cohort of patients. In particular, it was found that a SALF score >0.55 provided an AUC of 0.970 and 0.920 to detect liver cirrhosis in the training and validation sets, respectively, with high sensitivity (95.0% and 90.2%) and specificity (90.0% and 87.0%). This model showed a diagnostic performance which was similar to the ELF score (AUC 0.690-0.990) (Xie et al., 2014. PloS One 9(4):e92772) and the Hepascore (0.730-0.850) (Huang et al., 2017. Liver nt 37(1):121-131). Furthermore, the SALF score showed a better performance in saliva than the combination of the same biomarkers in serum.

The SALF score is proposed to be particularly useful for patients for whom a liver biopsy would pose a high risk, as well as for children due to its minimally invasive nature. In comparison to other non-invasive sampling methods such as blood, feces and urine, saliva presents the advantage of better patient compliance (Franco-Martinez et al., Saliva as a Non-invasive Sample: Pros and Cons, in: A. Tvarijonaviciute, S. Martinez-Subiela, P. Lopez-Jornet, E. Lamy (Eds.), Saliva in Health and Disease: The Present and Future of a Unique Sample for Diagnosis, Springer International Publishing, Cham, 2020, pp. 49-65). Furthermore, specific training is not necessary for collection, which can be performed at home by the patient, and further facilitates sequential sampling (Yoshizawa et al., 2013. Clin Microbiol Rev 26(4):781-791). Finally, the introduction of saliva diagnosis for liver disease would significantly improve health care in rural and geographically isolated regions. A study conducted by Roberts et al. showed that the prevalence of NAFLD in rural regions of Australia is considerably higher (36%) than the average prevalence in white populations (25%) (Roberts et al., 2021. supra). A similar disparity was observed in the United States, where patients with end-stage liver disease admitted to hospitals in rural areas had over twice the odds of experiencing in-hospital mortality compared to urban hospitals (Ross et al., 2019. Liver Transplant 25(9):1321-1332). In this context, saliva presents the advantage of being stored without the need for special laboratory equipment (Franco-Martinez et al., 2020. supra) collected into stabilizing buffers for short-term transport (Franco-Martinez et al., 2020. supra; Groschl et al., 2008. J Pharm Biomed Anal 47(3):478-486) and, more recently, applied to point-of-care devices (Khan et al., 2017. Diagnostics 7(3):39).

The present disclosure provides salivary biomarkers for diagnosis of liver fibrosis and demonstrates that specific serum biomarkers can be detected in saliva samples, and are significantly increased in patients with liver cirrhosis compared to healthy individuals and patients with underlying liver disease. Additionally, a saliva-based score for diagnosis of or screening for liver fibrosis, including liver cirrhosis, is disclosed, which inter alia is proposed as a screening tool for cirrhosis in high-risk asymptomatic populations, and for reducing the proportion of patients who progress to liver failure and/or cancer.

### Materials and Methods

### Participants

The study complies with the 2013 Declaration of Helsinki (Holm, Declaration of Helsinki, International encyclopedia of ethics (2013) 1-4). Human research ethics approval was obtained from the Greenslopes Research and Ethics Committee (approval number: 18/06), Queensland University of Technology (approval number: 2000000690) and The University of Queensland (approval number: 2000000690). Patients were recruited from the Greenslopes Private Hospital, Brisbane, Australia. All patients provided written informed consent prior to inclusion in the study.

For the training cohort, 10 liver cirrhosis patients (LC), 10 chronic liver disease patients without fibrosis (NF), 10 patients with intermediate degrees of fibrosis (IF), and 10 healthy controls (HC) were recruited. Liver fibrosis and/or cirrhosis were assessed using transient elastography (TE, FibroScan 502-, Echosens, France) by a trained operator. To obtain a liver stiffness measurement (LSM), a probe was placed in the intercostal space in the right lobe of the patient. Patients were recommended to refrain from eating before the examination. LSM results with at least 10 valid readings and an interquartile range (IQR) of less than 30% of the median LSM value were included (Lucidarme et al., 2009. Hepatology 49(4):1083-1089). The absence of fibrosis in the HC and NF cohorts was determined by an LSM 7.0 kPa, and liver cirrhosis was determined by an LSM >14.0 kPa (Castera et al., 2008. J Hepatol 48(5):835-47). Patients with LSM values between 7.0 kPa and 12.0 kPa were classified as having intermediate fibrosis.

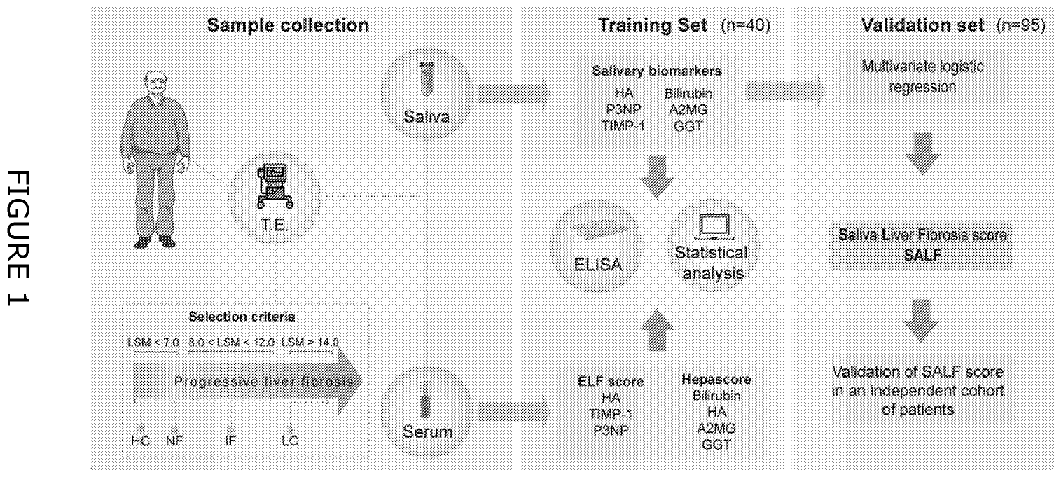

The validation cohort was composed of 95 individuals, classified as: 14 healthy controls (HC), 40 patients with non-fibrotic liver conditions (NF), 10 with intermediate fibrosis (IF) and 31 liver cirrhosis patients (LC) (FIG. 1).

### Sample Collection

Serum and salivary concentrations of six frequently used serum biomarkers for liver cirrhosis were measured in paired serum and saliva samples: hyaluronic acid (HA), tissue inhibitor of metalloproteinase 1 (TIMP-1), procollagen III amino-terminal propeptide (P3NP), γ—glutamyl transferase (GGT), total bilirubin, and α-2-macroglobulin (A2MG).

The following commercially available ELISA kits were used to quantify the concentration of biomarkers in blood and saliva samples: Human TIMP-1 DuoSet (R&D Systems, Minneapolis, MN, USA, cat #:DY970); Hyaluronan DuoSet (R&D Systems, Minneapolis, MN, USA Cat #:DHYALO); Human α-2-macroglobulin (R&D Systems, Minneapolis, MN, USA Cat #:DY1938); Human Procollagen Type III N-Terminal Propeptide (MyBioSource, San Diego, CA, USA, cat #:MBS045955). Total bilirubin and GGT were quantified using colorimetric assays (Bilirubin Assay Kit, Abcam, Cambridge, UK, cat #:ab235627; Gamma Glutamyl Transferase (GGT) Assay Kit, Abcam, Cambridge, UK, cat #ab241029). All measurements were performed according to manufacturers' instructions.

### Blood Fibrosis Test

The Enhanced Liver Fibrosis (ELF) score was calculated based on the algorithm proposed by Parkes et al (Parkes etal., 2011. J Viral Hepat 18(1):23-31). The Hepascore values were obtained using the logistic regression model proposed by Adams et al. [32]. The fibrosis-4 (FIB-4) and the AST-to-platelet ratio index (APRI) were determined using the patients laboratory measurements, as previously described (Sterling et al., 2006. Hepatology 43(6):1317-1325; Wai et al., 2003. Hepatology 38(2):518-526.).

### Development and Validation of the Salivary Biomarker Score

The diagnostic accuracy of the biomarkers was evaluated using receiver operating characteristic (ROC) curve analysis. A logistic regression predictive model was applied to the three biomarkers with the highest area under the curve (AUC) to calculate an individual score for each sample. This model, named Saliva Liver Fibrosis (SALF) score, was then validated using an independent cohort of patients with different degrees of liver fibrosis (n=40). The optimal cut-off values were determined based on the highest Youden's index (W. J. Youden, 1950. Cancer 3(1):32-35).

### Statistical Analysis

The software GraphPad Prism 9 version (GraphPad Software Inc., La Jolla, CA, USA) and R (R Development Core Team, Vienna, Austria) were used for statistical analysis. Continuous variables were tested for normality using the Shapiro-Wilk normality test. Kruskal-Wallis test was performed on data with non-normal distribution to compare values between multiple groups. One-way ANOVA was performed for group comparison in data with a normal distribution. GraphPad was used to generate standard curves for the ELISA assays by plotting the absorbance in the γ-axis and concentration of the analyte in the x-axis. The concentration of the analyte in the samples was deduced from the standard curve using a nonlinear regression equation. Biomarker concentration is expressed as mean t SD. Correlation studies were executed using Spearman's correlation test.

### Example 2

### Optimization of Salf Algorithm

An updated version of the SALF score was developed with the addition of 80 patients samples and the optimization of the score. Patients (n=206) were divided into a training (n=103) and a validation cohort (n=103), according to Table 6. The concentration of HA, TIMP-1 and A2MG was elevated in the saliva of liver cirrhosis and liver fibrosis patients both in the training and validation cohorts (FIG. 6).

TABLE 6
Patients cohorts for the optimization of the SALF score.

| Cohort | Training | Validation |
|---|---|---|
| Healthy control | 12 | 12 |
| Liver disease | 32 | 32 |
| Fibrosis | 27 | 27 |
| Cirrhosis | 32 | 32 |
| Total | 103 | 103 |

The diagnostic performance of the markers was evaluated by generating a ROC curve (Table 7). Overall, A2MG showed the best performance for the diagnosis of cirrhosis both against healthy controls (AUROC 0.743) and liver disease patients without any fibrosis (AUROC 0.751). The main goal of the study is to detect significant fibrosis (fibrosis+cirrhosis) against healthy controls and liver disease patients. For this comparison, TIMP-1, HA, and A2MG showed an AUROC of 0.635, 0.679, and 0.672, respectively, with sensitivity values ranging from 51.6% to 54.8% and specificity between 72.7% and 79.2%.

TABLE 7
ROC analysis of HA, TIMP-1 and A2MG
as fibrosis/cirrhosis biomarkers.

| | | | | |
|---|---|---|---|---|
| AUROC | Sens | Spec | NPV | PPV |

Cirrhosis vs Healthy

| | | | | | |
|---|---|---|---|---|---|
| TIMP1 | 0.686 | 50.0 | 83.3 | 37.0 | 89.5 |
| HA | 0.792 | 61.8 | 100.0 | 48.0 | 100.0 |
| A2MG | 0.743 | 61.8 | 83.3 | 43.5 | 91.3 |

Cirrhosis vs Liver Disease

| | | | | | |
|---|---|---|---|---|---|
| TIMP1 | 0.733 | 76.5 | 62.5 | 71.4 | 68.4 |
| HA | 0.718 | 85.3 | 62.5 | 80.0 | 70.7 |
| A2MG | 0.751 | 52.9 | 87.5 | 63.6 | 81.8 |

Cirrhosis + Fibrosis vs Healthy + Liver disease

| | | | | | |
|---|---|---|---|---|---|
| TIMP1 | 0.635 | 54.8 | 72.7 | 53.3 | 73.9 |
| HA | 0.679 | 54.8 | 86.4 | 57.6 | 85.0 |
| A2MG | 0.672 | 51.6 | 79.2 | 53.8 | 78.0 |

Sens: Sensitivity (%); Spec: Specificity (%); PPV: Positive Predictive Value (%); NPV: Negative Predictive Value (%).

The same methodology applied to develop the original SALF score was applied to the new training cohort. A logistic regression model was applied to generate a scoring system to detect fibrosis and cirrhosis against the non-fibrotic individuals. The following algorithm was generated:

$$y = EXP [ - 1.78007 + ( HA \times 0.1132 ) + ( TIMP 1 \times 0.0431 ) + ( A 2 MG \times 0.05163 ) ]$$

$$SALF = \frac{y}{1 + y}$$

The performance of the SALF score in a training and validation cohort is summarized in Table 8. The SALF score demonstrated the best performance for the detection of cirrhosis versus healthy controls, with AUROC values of 0.933 and 0.875 in a training and validation cohort, respectively. For the detection of significant fibrosis (Fibrosis+Cirrhosis cohorts), the SALF score had an overall AUROC of 0.813 with 70.6% sensitivity and 86.2% specificity.

TABLE 8
ROC analysis of HA, TIMP-1 and A2MG
as fibrosis/cirrhosis biomarkers.

| | | | | |
|---|---|---|---|---|
| AUROC | Sens | Spec | NPV | PPV |

Cirrhosis vs Healthy

| | | | | | |
|---|---|---|---|---|---|
| Training | 0.933 | 87.1 | 91.7 | 73.3 | 96.4 |
| Validation | 0.875 | 70.6 | 91.7 | 52.4 | 96.0 |
| Total | 0.895 | 72.3 | 95.8 | 56.1 | 97.9 |

Cirrhosis vs Liver Disease

| | | | | | |
|---|---|---|---|---|---|
| Training | 0.918 | 74.2 | 100.0 | 79.5 | 100.0 |
| Validation | 0.844 | 79.4 | 81.2 | 78.8 | 81.8 |
| Total | 0.845 | 76.9 | 85.7 | 78.3 | 84.7 |

Cirrhosis + Fibrosis vs Healthy + Liver disease

| | | | | | |
|---|---|---|---|---|---|
| Training | 0.887 | 71.9 | 100 | 72.9 | 100.0 |
| Validation | 0.760 | 71.0 | 75.0 | 64.7 | 80.0 |
| Total | 0.813 | 70.6 | 86.2 | 68.2 | 87.5 |

### Example 3

### Biomarker Discovery Through Untargeted Mass Spectrometry (Swath-Ms)

An untargeted mass spectrometry screening biomarker discovery was performed in the saliva of healthy (n=10), liver disease (n=10), and cirrhosis (n=10) patients. Three proteins were significantly altered in the saliva of liver cirrhosis patients: carbonic anhydrase 1 (CA1), importin subunit alpha-3 (also known as karyopherin subunit alpha-4) (KPNA3) and 6-phosphogluconolactonase (PGLS) (FIG. 7A). This was further confirmed in a validation cohort (n=94) composed of 19 healthy controls, 45 liver disease and 30 liver cirrhosis patients (FIG. 7B).

To confirm the mass spectrometry results, the proteins of interest were detected using western blot analysis (FIG. 8A). The three proteins were detected in individual samples from healthy controls and patients with liver disease and cirrhosis. Similar to the SWATH data, CA1 and PGLS showed a higher abundance in the saliva of liver cirrhosis patients compared to saliva from healthy controls and liver disease patients. Immunohistochemistry staining of liver sections obtained from Mdr2−− mice was also performed. This mouse model, commonly used in the study of liver fibrosis, has a disruption of the Mdr2 gene which leads to a complete absence pf phosphatidylcholine from the bile, resulting in progressive liver damage (Ikenaga et al. Am J Pathol. 2015 February;185(2):325-34). Staining performed on knockout animals with advanced fibrosis and healthy wild-type mice showed an increase in CA1 and PGLS in the fibrotic animals (FIG. 5B). This was not observed for KPNA3. These results demonstrate not only that CA1, PGLS, and KPNA3 can be accurately detected in saliva, but also that CA1 and PGLS might play a role in the fibrogenic process.

To further improve the accuracy of the proposed biomarkers for the detection of liver fibrosis and cirrhosis, a diagnostic score was generated using logistic regression, as follows:

$$y = EXP [ - 2.139255264 + ( - 649329.5 \; 654 \times KPNA 3 ) + ( 180500.0745 \times PGLS ) + ( A 51528.8054 \times CA 1 ) ]$$

$$Score = \frac{y}{1 + y}$$

The combination resulted in significantly higher scores in the liver cirrhosis group (median: 0.836) compared to patients without fibrosis (median: 0.02) and healthy controls (median: 0.006). The diagnostic performance of the panel is summarized in Table 9.

TABLE 9
ROC analysis of the panel combining CA1, PGLS and KPNA3.

| | | | | |
|---|---|---|---|---|
| AUROC | Sens | Spec | NPV | PPV |

Cirrhosis vs Healthy

| | | | | | |
|---|---|---|---|---|---|
| Training | 0.990 | 100 | 98 | 100.0 | 100.0 |
| Validation | 0.749 | 63.3 | 84.2 | 59.3 | 86.4 |
| Total | 0.804 | 70.0 | 89.7 | 68.4 | 90.3 |

Cirrhosis vs Liver Disease

| | | | | | |
|---|---|---|---|---|---|
| Training | 0.910 | 90.0 | 90.0 | 90.0 | 90.0 |
| Validation | 0.747 | 63.3 | 82.2 | 77.1 | 70.4 |
| Total | 0.769 | 70.0 | 83.6 | 79.3 | 75.7 |

Cirrhosis vs Healthy + Liver disease

| | | | | | |
|---|---|---|---|---|---|
| Training | 0.950 | 90.0 | 95.0 | 95.0 | 90.0 |
| Validation | 0.748 | 63.3 | 82.8 | 82.8 | 61.3 |
| Total | 0.781 | 70.0 | 85.7 | 85.7 | 70.0 |

### Example 4

### Combination of 6 Salivary Biomarkers as a Multi-Biomarker Panel

The diagnostic performance of a multi-biomarker panel consisting of HA, TIMP-1, A2MG, CA1, PGLS and KPNA3 was analyzed using saliva samples from a subset of patients. This cohort consisted of 20 healthy controls, 45 liver disease patients and 30 liver cirrhosis. The results are summarized in Table 10.

TABLE 7
ROC analysis of multi-biomarker panel.

| | | | | |
|---|---|---|---|---|
| AUROC | Sens | Spec | NPV | PPV |

Cirrhosis vs Healthy

| | | | | | |
|---|---|---|---|---|---|
| HA + TIMP1 + A2MG | 0.929 | 96.0 | 83.3 | 93.8 | 88.9 |
| CA1 + PGLS + KPNA3 | 0.820 | 84.0 | 72.2 | 76.5 | 80.8 |
| 6 markers | 0.993 | 96.0 | 100.0 | 94.7 | 100.0 |

Cirrhosis vs Liver Disease

| | | | | | |
|---|---|---|---|---|---|
| HA + TIMP1 + A2MG | 0.837 | 92.0 | 77.4 | 92.3 | 76.7 |
| CA1 + PGLS + KPNA3 | 0.827 | 80.0 | 74.2 | 82.1 | 71.4 |
| 6 markers | 0.874 | 94.1 | 71.4 | 93.8 | 72.7 |

Cirrhosis vs Healthy + Liver disease

| | | | | | |
|---|---|---|---|---|---|
| HA + TIMP1 + A2MG | 0.871 | 92.0 | 79.6 | 95.1 | 69.7 |
| CA1 + PGLS + KPNA3 | 0.827 | 80.0 | 74.2 | 82.1 | 71.4 |
| 6 markers | 0.945 | 88.0 | 91.8 | 93.8 | 84.6 |

### Example 5

Detection of Ca1, Pgls and Kpna3 in Serum and Correlation with Salivary Levels

Paired serum and saliva samples were analyzed to investigate the relationship between the concentrations of CA1, PGLS and KPNA3 in blood and saliva. No significant differences were observed in the concentration of the proteins between healthy controls, liver disease, liver fibrosis and cirrhosis patients (FIG. 9A). However, the analysis of a larger number of samples is necessary. A Spearman's correlation analysis resulted in a significant moderate correlation between (r=0.550, p=0.03) serum and salivary concentrations of PGLS. This was not observed for CA1 (r=0.257, p=0.06) and KPNA3 (r=−0.04, p=0.87) (FIG. 9B).

Overall, the new data demonstrate that the SALF score was optimized with the addition of new samples into the analysis. Importantly, with the development of the new algorithm, a decrease in the diagnostic performance of the SALF score was not observed, as shown by the ROC analysis of the total cohorts.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

What is claimed is:

1. A method for determining an indicator used in assessing a likelihood of a subject having a presence, absence or degree or severity of liver fibrosis, the method comprising, consisting or consisting essentially of:
(1) determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS); and
(2) determining the indicator using the biomarker value(s)s.

2. A method for determining an indicator used in assessing a likelihood that liver cirrhosis is present, absent or developing in a subject, the method comprising, consisting or consisting essentially of:
(1) determining a biomarker value for at least one (e.g., 1, 2, 3, 4, 5, 6, etc.) biomarker in a saliva sample obtained from the subject, wherein a respective biomarker value is indicative of a level of a corresponding biomarker in the sample, and wherein the at least one biomarker is selected from A2MG, HA, TIMP1, CA1, KPNA3, and PGLS; and
(2) determining the indicator using the biomarker value(s).

3. The method of claim 1 or claim 2, wherein biomarker values are determined for each of A2MG, HA and TIMP1 and the indicator is determined using those biomarker values.

4. The method of claim 1 or claim 2, wherein biomarker values are determined for each of CA1, KPNA3, and PGLS and the indicator is determined using those biomarker values.

5. The method of claim 1 or claim 2, wherein biomarker values are determined for each of A2MG, HA, TIMP1, CA1, KPNA3, and PGLS and the indicator is determined using those biomarker values.

6. The method of any one of claims 1 to 5, further comprising applying a function to biomarker values to yield at least one functionalized biomarker value and determining the indicator using the at least one functionalized biomarker value.

7. The method of any one of claims 1 to 6, further comprising combining the biomarker values to provide a composite score and determining the indicator using the composite score.

8. The method of any one of embodiments 1 to 7, further comprising analyzing the biomarker value(s with reference to a corresponding reference biomarker value range or cut-off values, to determine the indicator.

9. The method of any one of embodiments 6, further comprising analyzing the functionalized biomarker value(s) with reference to a corresponding functionalized biomarker value range or cut-off values, to determine the indicator.

10. The method of any one of embodiments 7, further comprising analyzing the composite score with reference to a corresponding reference composite score range or cut-off values, to determine the indicator.

11. A method for monitoring liver fibrosis status or treatment of a subject, the method comprising, consisting or consisting essentially of:
(1) determining a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from A2MG, HA, TIMP1, CA1, KPNA3 and PGLS;
(2) determining a first indicator using the biomarker value(s);
(3) determining a biomarker value for each of the at least one biomarkers, for which biomarker values were determined in the first saliva sample, in a second saliva sample obtained from the subject, wherein a respective biomarker value is indicative of a level of a corresponding biomarker in the second sample;
(4) determining a second indicator using the biomarker value(s); and
(5) comparing the first indicator with the second indicator, thereby monitoring the liver fibrosis status or treatment of the subject.

12. An apparatus for determining an indicator used in assessing a likelihood of a subject having a presence, absence or degree or severity of liver fibrosis, the apparatus comprising at least one electronic processing device that:
determines a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS); and
determines the indicator using the derived biomarker value(s).

13. An apparatus for determining an indicator used in assessing a likelihood that liver cirrhosis is present, absent or developing in a subject, the apparatus comprising at least one electronic processing device that:
determines a biomarker value for at least one (e.g., 1, 2, 3, 4, 5, 6, etc.) biomarker in a first saliva sample obtained from the subject, wherein a respective biomarker value is indicative of a level of a corresponding biomarker in the sample, and wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS); and
determines the indicator using the derived biomarker value(s).

14. A composition comprising a mixture of a saliva sample obtained from a subject, and for at least one (e.g., 1, 2, 3, 4, 5, 6, etc.) biomarker an antibody or antigen-binding fragment that binds specifically to the biomarker, wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS).

15. A method for inhibiting the development or progression of liver fibrosis in a subject, the method comprising exposing the subject to a treatment regimen for treating liver fibrosis at least in part on the basis that the subject is determined by the indicator-determining method of any one of claims 1 and 3 to 6 as having a likelihood of a presence or degree or severity of liver fibrosis.

16. A method for inhibiting the development or progression liver cirrhosis in a subject, the method comprising exposing the subject to a treatment regimen for treating liver cirrhosis at least in part on the basis that the subject is determined by the indicator-determining method of any one of claims 2 to 6 as having a likelihood of a presence or development of liver cirrhosis.

17. A kit for determining an indicator used in assessing a likelihood of a subject having a presence, absence or degree or severity of liver fibrosis, or a likelihood that liver cirrhosis is present, absent or developing in a subject, the kit comprising: for at least one biomarker an antibody or antigen-binding fragment that binds specifically to the biomarker, wherein the at least one biomarker is selected from α-2-macroglobulin (A2MG), hyaluronic acid (HA), tissue inhibitor matrix metalloproteinase 1 (TIMP1), carbonic anhydrase 1 (CA1), importin subunit alpha-4 (also known as karyopherin subunit alpha-3) (KPNA3) and 6-phosphogluconolactonase (PGLS).
